(19) United States
(12) Patent Application Publication
(10) Pub. No.: US 2026/0253725 A1
(43) Pub. Date: Aug. 27, 2026

(54) MEDICAL ASSISTANCE DEVICE, MEDICAL ASSISTANCE METHOD, AND MEDICAL ASSISTANCE PROGRAM

(71) Applicant: THE UNIVERSITY OF TOKYO, Tokyo (JP)

(72) Inventor: Akiko KISHI, Tokyo (JP)

(21) Appl. No.: 18/725,490

(22) PCT Filed: Dec. 9, 2022
(86) PCT No.: PCT/JP2022/045444

§ 371 Date: Jun. 28, 2024

(30) Foreign Application Priority Data
Dec. 28, 2021 (JP) 2021-214424

(51) Int. Cl. G16H 50/20; G16H 10/20; G16H 10/60; G16H 20/30; G16H 50/30

(52) U.S. Cl. CPC G16H 50/20; G16H 10/20; G16H 10/60; G16H 20/30; G16H 50/30

(57) ABSTRACT

Provided is a medical assistance device including an acquisition unit configured to acquire personal data including examination data and medical interview data of a user, and an output unit configured to determine a disease state of the user for an individual disease forming a complex disease, based on the personal data, and configured to output an improvement target to be achieved by the user to improve a complex disease state, when a combination of the disease states of the individual diseases or a combination of the disease state of the individual disease and the personal data is the complex disease state.

### TECHNICAL FIELD

The present disclosure relates to a medical assistance device, a medical assistance method, and a medical assistance program.

### BACKGROUND ART

Currently, a system for improving a health state of a user is known. For example, Japanese Patent Application Laid-Open No. 2021-176053 discloses a health management device which can propose a measure for maintaining or improving the health state of the user, based on a nutritional value of a food taken by the user and the health state of the user.

In addition, at present, medical devices or medical programs which aim to assist a treatment of an individual disease such as diabetes have been approved and provided in Europe and the like. However, since a target of the medical devices or the medical programs is only the individual disease, it is difficult to assist a treatment of a patient in which a plurality of diseases are complexed, for example, a metabolic syndrome.

### SUMMARY OF INVENTION

Therefore, an object of the present disclosure is to provide a technology which can assist a treatment of a user even in a state where a plurality of diseases are complexed.

According to one aspect of the present disclosure, there is provided a medical assistance device including an acquisition unit configured to acquire personal data including examination data and medical interview data of a user, and an output unit configured to determine a disease state of the user for an individual disease forming a complex disease, based on the personal data, and configured to output an improvement target to be achieved by the user to improve a complex disease state, when a combination of the disease states of the individual diseases or a combination of the disease state of the individual disease and the personal data is the complex disease state.

According to the present disclosure, it is possible to provide a technology which can assist a treatment of a user even in a state where a plurality of diseases are complexed.

### BRIEF DESCRIPTION OF DRAWINGS

FIG. 1 is a view showing an example of a medical assistance system according to the present embodiment.

FIG. 2 is a flowchart showing an outline of a process executed by the medical assistance system.

FIG. 3 is a view showing an example of personal data.

FIG. 4 is a view showing an example of a case where a complex disease state is determined based on a determination method 1.

FIG. 5 is a view showing an example of a case where the complex disease state is determined based on a determination method 2.

FIG. 6 is a schematic view showing a flow of medical assistance using the medical assistance system.

FIG. 7 is a view showing a hardware configuration example of a medical assistance device, a patient terminal, and a doctor terminal.

FIG. 8 is a view showing a functional block configuration example of the medical assistance device.

FIG. 9A is a view showing an example of patient information, individual disease definition information, complex disease/target definition information, questionnaire definition information, and score definition information.

FIG. 9B is a view showing an example of advice definition information.

FIG. 10 is a view showing an example of a trained model.

FIG. 11 is a view showing a functional block configuration example of the patient terminal.

FIG. 12 is a view showing a functional block configuration example of the doctor terminal.

FIG. 13 is a sequence diagram showing an example of a process procedure performed by the medical assistance system.

FIG. 14 is a sequence diagram showing an example of a process procedure performed by the medical assistance system.

FIG. 15 is a view showing a specific example of examination data.

FIG. 16 is a view showing a specific example of medical interview data.

FIG. 17A is a view showing a specific example of the individual disease definition information.

FIG. 17B is a view showing a specific example of the individual disease definition information.

FIG. 18 is a view showing a specific example of the complex disease/target definition information.

FIG. 19 is a view showing an example of behavior target definition information.

FIG. 20 is a view showing a screen display example of the patient terminal.

FIG. 21 is a view showing a screen display example of the patient terminal.

FIG. 22 is a view showing a screen display example of the patient terminal.

FIG. 23 is a view showing a screen display example of the patient terminal.

FIG. 24 is a view showing a screen display example of the patient terminal.

FIG. 25 is a view showing a screen display example of the patient terminal.

FIG. 26 is a view showing a screen display example of the patient terminal.

FIG. 27 is a view showing a screen display example of the patient terminal.

FIG. 28 is a view showing a screen display example of the doctor terminal.

FIG. 29 is a view showing a screen display example of the doctor terminal.

FIG. 30 is a view showing a screen display example of the doctor terminal.

FIG. 31 is a view showing experimental data.

### DESCRIPTION OF EMBODIMENTS

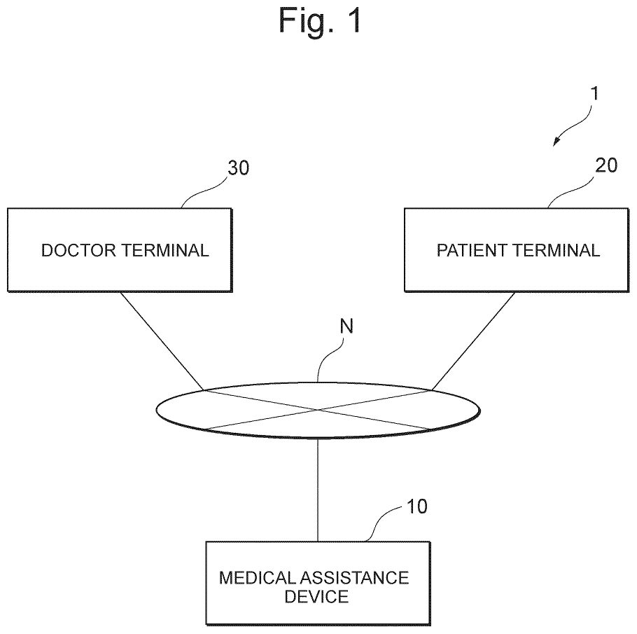

Embodiments of the present invention will be described with reference to the accompanying drawings. In each drawing, elements provided with the same reference numerals have the same or similar configurations.

<System Configuration> FIG. 1 is a view showing an example of a medical assistance system 1 according to the present embodiment. The medical assistance system 1 is a system for assisting a disease treatment of a patient, and includes a medical assistance device 10, a patient terminal 20 (first terminal), and a doctor terminal 30 (second terminal). The medical assistance device 10, the patient terminal 20, and the doctor terminal 30 are connected to each other via a wireless or wired communication network N, and can communicate with each other. The number of the patient terminals 20 and the doctor terminals 30 may be one, or two or more.

The medical assistance system 1 is a system in which a patient (may be referred to as a “user”) who has a plurality of diseases is enabled to understand a disease state of the patient himself/herself, and which aims to assist recovery from the disease state by promoting a behavior change of the patient himself/herself. For example, the medical assistance system 1 may have a plurality of functions including the following: 1. Function of determining the disease state of the patient; 2. Function of indicating the disease state of the patient to a doctor; 3. Function of proposing an improvement target to be achieved to improve the disease state to the doctor and receiving settings of the improvement target to be achieved by the patient; 4. Function of indicating the disease state of the patient himself/herself and presenting the improvement target to be achieved to the patient; 5. Function of receiving settings of a behavior target (may be referred to as a “challenge”) to be handled to achieve the improvement target from the patient; 6. Function of displaying a score relating to an achievement degree of the behavior target to be handled by the patient, based on an answer to questionnaire relating to a daily behavior target which is received from the patient.

The medical assistance device 10 has a function of performing determination of the disease state of the patient, proposal of the improvement target, setting and receiving of the daily behavior target, or the like, and displaying various types of information relating to the patient on screens of a patient terminal 20 and a doctor terminal 30.

The patient terminal 20 is a terminal used by the patient, and has a function of displaying information received from the medical assistance device 10 and a function of receiving various inputs from the patient. For example, the patient terminal 20 may be a smartphone, a tablet terminal, a personal computer, or the like.

The doctor terminal 30 is a terminal used by the doctor, and has a function of displaying information received from the medical assistance device 10 and a function of receiving various inputs from the doctor. It is assumed that the doctor terminal 30 is a personal computer or the like installed in a medical institution, but without being limited thereto, the doctor terminal 30 may be the smartphone or the tablet terminal, for example. The medical assistance device 10 has a Web server function, and may be configured to realize information display and input reception on the doctor terminal 30 in such a manner that a Web browser installed in the doctor terminal 30 accesses the medical assistance device 10. Alternatively, the medical assistance device 10 may be configured to realize information display and input reception on the doctor terminal 30 in such a manner that a dedicated application installed in the patient terminal 20 accesses the medical assistance device 10.

The patient terminal 20 may include a wearable device worn by the patient. The medical assistance system 1 may be configured to acquire and store various types of data (hereinafter, referred to as “biological information”) relating to the patient from the wearable device. For example, the patient may wear the wearable device to transmit various types of biological information (heart rate, number of steps, exercise amount, or the like) to the medical assistance device 10, and may be configured to set the behavior target, or to refer to the score relating to the achievement degree of the behavior target by using the smartphone.

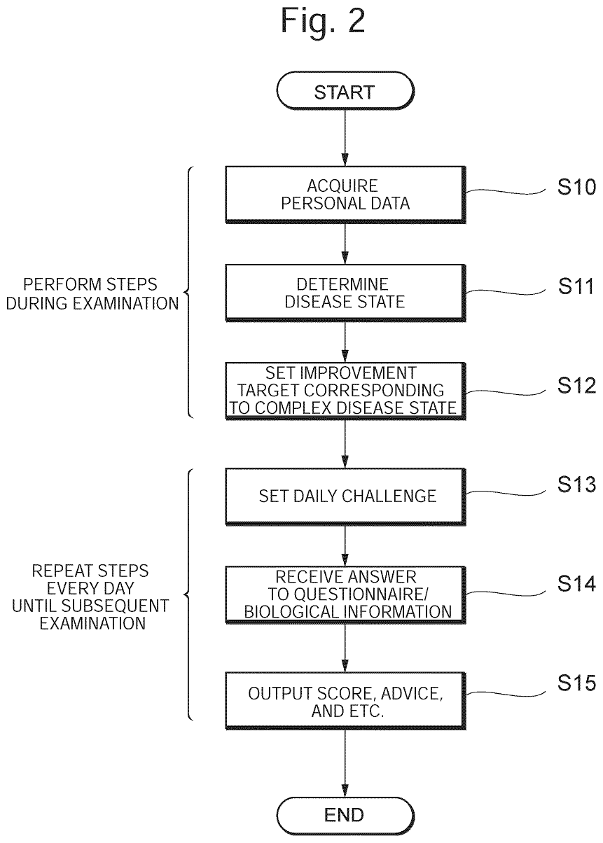

FIG. 2 is a flowchart showing an outline of a process executed by the medical assistance system 1. First, the medical assistance device 10 acquires the personal data of the patient (S10). The personal data includes any one or both of examination data obtained by examining a body state of the patient in a medical institution or the like and medical interview data obtained by asking a question to the patient. For example, the examination data may include data obtained by measuring a body of the patient, such as a body height, a body weight, body fat, BMI, and abdominal circumference (body measurement data), data obtained by examining blood (blood examination data), data obtained by examining urine (urine examination data), and data or the like obtained by examining a state of a body site, such as an organ and a blood vessel (organ data).

In addition, for example, the blood examination data may include lipid-related data such as neutral fat, HDL cholesterol, LDL cholesterol, and non-HDL cholesterol, liver/pancreatic function-related data such as AST (GOT), ALT (GPT), and γ-GPT, kidney function-related data such as creatinine, anemia-related data such as hematocrit value, hemoglobin amount, white blood cell amount, and red blood cell amount, blood pressure-related data such as systolic blood pressure and diastolic blood pressure, and general blood data including blood glucose-related data such as fasting blood glucose (potential difference method), HbA1c (NGSP value), or a uric acid value. In addition, for example, the urine examination data may include data such as urine sugar, urine protein, urine occult blood, and the like.

For example, the medical interview data may include data relating to a disease suffered by the patient in the past (past history), data relating to a disease in which it is previously found out that the disease is currently suffered by the patient (for example, disease name), past and current data relating to a disease of a family or a close relative (family history), data relating to drug history (presence or absence of drug use in the past and at present, name of specific drug taken or being taken by the patient, and the like), data relating to a social history (educational background, annual income, family structure, and the like), smoking information (presence or absence of smoking habit in the past and at present, smoking amount, smoking duration, presence or absence of willingness to improve smoking habit, and the like), alcohol drinking information (presence or absence of drinking experience, alcohol drinking amount, and specific contents of drinking habit such as type and duration or the like), dietary habit (number of meals, meal contents, presence or absence of eating-out, presence or absence of willingness to improve contents of the meal, and the like), activity amount (presence or absence of daily exercise habit, specific exercise amount, presence or absence of willingness to improve frequency and activity contents, and the like), information relating to body weight change, sleeping information (information relating to quality and quantity of sleeping, presence or absence of willingness to improve sleeping habit, and the like), and data relating to lifestyle such job contents, attendance information, hobby, preference, stress, and the like. The past history, the disease name, and the family history are collectively referred to as a “clinical history.” The present embodiment is not limited to a case where the clinical history includes all of the past history, the disease name, and the family history. Those which include at least one of the past history, the disease name, and the family history may be referred to as the clinical history.

Subsequently, the medical assistance device 10 determines a disease state suffered by the patient, based on the acquired personal data of the patient (S11). More specifically, the medical assistance device 10 determines the presence or absence of an individual disease (hereinafter, referred to as an “individual disease”) suffered by the patient from the personal data of the patient. The individual disease means one disease, and for example, the individual disease includes lifestyle habit-related diseases such as obesity, diabetes, hypertension, dyslipidemia, fatty liver, cardiovascular disease, cerebrovascular disease, osteoporosis, dementia, periodontal disease, alcoholic hepatitis, non-alcoholic fatty liver disease, liver cirrhosis, hyperuricemia, gastric ulcer, peripheral neuropathy, allergy, and the like. In addition to these diseases, the individual disease includes various diseases such as cancer, renal failure, chronic obstructive pulmonary disease, arthritis, lumbago, cataract, glaucoma, sleep apnea syndrome, and the like. Next, when the patient corresponds to a plurality of individual diseases defined in advance as a combination pattern, or when the patient corresponds to conditions of the plurality of individual diseases and the personal data which are defined in advance as a combination pattern, the medical assistance device 10 determines that the patient is in a “complex disease state.” In addition, when the patient does not correspond to the plurality of individual diseases defined in advance as the combination pattern, or when the patient corresponds to the plurality of individual diseases defined in advance as the combination pattern but does not correspond to the condition of the personal data, the medical assistance device 10 determines that the patient is not in the “complex disease state.” That is, the “complex disease state” means a state corresponding to the plurality of individual diseases defined in advance as the combination pattern, or a state corresponding to the condition of the plurality of individual diseases and the personal data which are defined in advance as the combination pattern. The “condition of the personal data” means that a predetermined item of the personal data satisfies a predetermined condition. For example, examples of the “condition of the personal data” include that an item indicating a gender of the personal data is a male, an item indicating an age of the personal data is equal to or older than XX years, an item indicating an abdominal circumference of the personal data is equal to or larger than XX cm, and the like.

For example, specific examples of the complex disease state include a metabolic syndrome. In a case of the metabolic syndrome, the “plurality of individual diseases defined in advance as the combination pattern” correspond to at least two or more of diabetes (blood glucose level is an abnormal value), hypertension (blood pressure is an abnormal value), and dyslipidemia (lipid is an abnormal value), and the “condition of the personal data” may be defined as that at least the abdominal circumference is equal to or larger than 85 cm (in a case of a male) or is equal to or larger than 90 cm (in a case of a female). In addition, in addition to the abdominal circumference, the “condition of the personal data” may be defined as that the condition satisfies diagnostic reference values of various diseases which can be acquired from the personal data. In accordance with the definition, the medical assistance device 10 determines that the patient is in a complex disease state (metabolic syndrome), when the patient suffers from two or more of diabetes, hypertension, and dyslipidemia, and when the patient is the male and has the abdominal circumference which is equal to or larger than 85 cm (equal to or larger than 90 cm in a case of the female). Instead of a determination reference in which the abdominal circumference is equal to or larger than 85 cm (in a case of the male) or is equal to or larger than 90 cm (in a case of the female), the metabolic syndrome may adopt a determination reference in which the patient suffers from obesity (more specifically, a state of visceral fat type obesity). In this case, the “plurality of individual diseases defined as a combination pattern in advance” in the metabolic syndrome may be defined as to correspond to at least two or more of diabetes, hypertension, or dyslipidemia in addition to the obesity (visceral fat type obesity state).

In addition, another example of the complex disease state includes a state where the plurality of individual diseases such as chronic individual diseases, for example, diabetes, hypertension, malignant neoplasm (cancer), heart disease (excluding hypertensive heart disease), or diseases such as cerebrovascular disease, and diseases such as anxiety, depressive state, pain, physical disorder, neurological disorder, and the like concurrently occur, although a specific disease name is not assigned at the moment. In this complex disease state, a treatment is independently performed in many cases. The number of drugs is likely to increase, and a drug taking pattern is likely to be complicated. In addition, since a treatment guideline generally targets a single disease, the treatment is fragmented, and a case of insufficient communication between doctors who treat respective diseases is likely to occur. Specifically, since the patient suffers from diabetes, it is reported that risks of developing stroke, acute myocardial infarction, heart failure, or depression are increased, and a risk of developing dementia is further increased. Therefore, it is conceivable that a certain patient is in a state of concurrently suffering from diabetes and depression or in a state of concurrently suffering from diabetes, depression, and dementia. In the present invention, this state where the plurality of individual diseases concurrently occur in this way or a risk of developing the diseases is high may be regarded as the complex disease state.

More specifically, the medical assistance device 10 may be configured to determine whether or not the patient corresponds to the complex disease state in accordance with the following method. Furthermore, the medical assistance device 10 may be configured to determine a risk value relating to the complex disease state. The risk value relating to the complex disease state may mean severity as the entire complex disease suffered by the patient, and may indicate a severe disease as the risk value is greater.

(Determination Method 1) The “plurality of individual diseases defined in advance as the combination pattern” may be defined as that the condition corresponds to at least the predetermined number or more of the individual diseases described above. The predetermined number may be 2, or may be 3 or more. In addition, when the patient suffers from the predetermined number or more of the individual diseases defined in advance as the combination pattern or has a high risk of developing the individual disease, the medical assistance device 10 may be configured to determine that the patient is in the complex disease state. In addition, the medical assistance device 10 may be configured to determine the risk value of the complex disease state, based on the number of the corresponding individual diseases in the plurality of individual diseases defined in advance as the combination pattern in the complex disease state. For example, when the number of the corresponding individual diseases is five, the medical assistance device 10 may be configured to determine this case as a risk 5. This case means that the risk increases as the number increases.

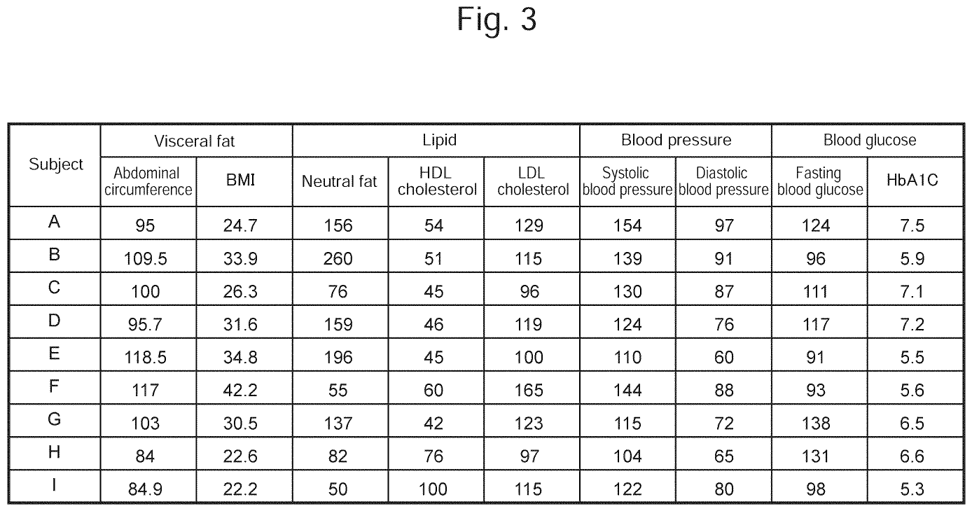

FIG. 3 is a view showing an example of the personal data. FIG. 3 shows an example of the personal data (examination data) obtained by examining subjects A to I for the abdominal circumference and BMI which are examination items relating to visceral fat, neutral fat which is an examination item relating to lipid, HDL cholesterol and LDL cholesterol, systolic blood pressure and diastolic blood pressure which are examination items relating to blood pressure, and fasting blood glucose and HbA1C which are examination items relating to blood glucose.

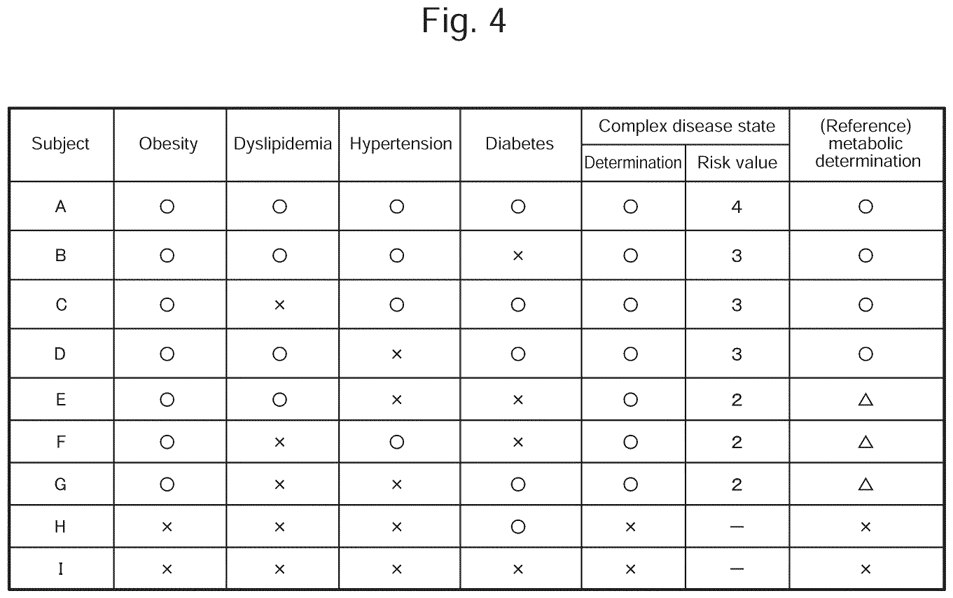

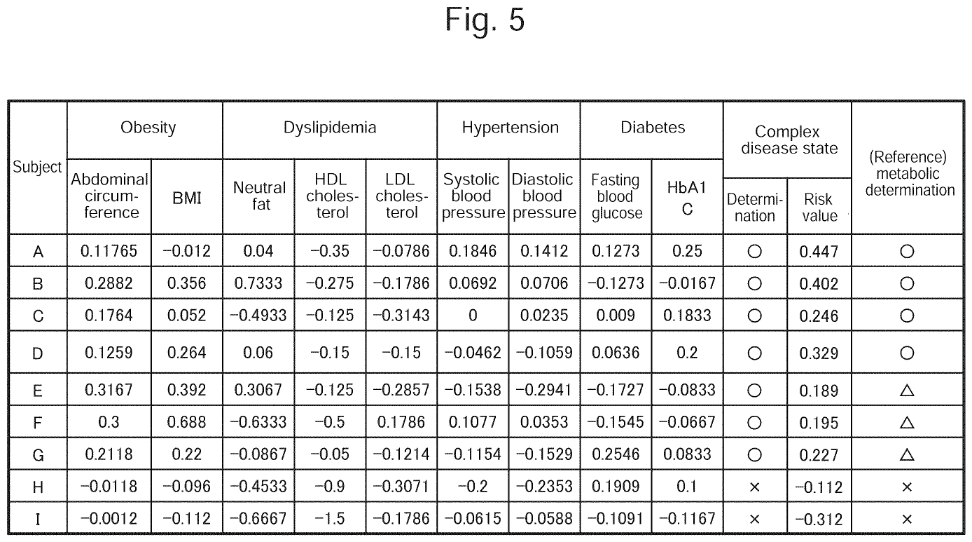

FIG. 4 is a view showing an example of a case where the complex disease state is determined based on a determination method 1. In addition, “∘” indicates that the patient corresponds to the complex disease state, and “x” indicates that the patient does not correspond to the complex disease state. In the example in FIG. 4 (the same applies to the example in FIG. 5), the medical assistance device 10 determines whether or not the patient corresponds to the metabolic syndrome as the complex disease state. For example, the patient whose personal data (abdominal circumference and BMI) relating to visceral fat does not satisfy a predetermined reference corresponds to obesity. The patient whose personal data (neutral fat and the like) relating to lipid does not satisfy a predetermined reference corresponds to dyslipidemia. The patient whose personal data (systolic blood pressure and the like) relating to blood pressure does not satisfy a predetermined reference corresponds to hypertension. The patient whose personal data (fasting blood glucose and the like) relating to blood glucose does not satisfy a predetermined reference corresponds to diabetes. In addition to determining whether or not the patient corresponds to each individual disease, the medical assistance device 10 may be configured to determine severity of each corresponding individual disease by using individual disease definition information 100b or a trained model (to be described below). The term “determination” indicates whether or not the patient corresponds to the complex disease state (here, metabolic syndrome). The term “(reference) metabolic determination” is described with reference to a determination result in which an actual doctor determines metabolic syndrome in accordance with references of the academic society. “∘” means that the patient corresponds to metabolic syndrome, and “Δ” means that the patient corresponds to a metabolic syndrome preliminary group.

In the example in FIG. 4, when the patient corresponds to two or more individual diseases in the plurality of individual diseases defined in advance as the combination pattern, the patient is determined as the complex disease state (metabolic syndrome), but the present disclosure is not limited thereto. For example, when the patient corresponds to three or more individual diseases, the patient may be determined as the complex disease state (metabolic syndrome).

(Determination Method 2) The medical assistance device 10 may be configured to calculate a risk for each examination item by using a reference value (may be referred to as a cutoff value) for one or more examination items relating to each of the plurality of individual diseases defined in advance as the combination pattern and examination data for the examination item obtained by examining the patient, and to determine that the patient is in the complex disease state when a value obtained by averaging the calculated risks for each examination item is equal to or greater than a predetermined threshold value. In addition, the medical assistance device 10 may be configured to determine that the patient is not in the complex disease state when the value obtained by averaging the risks for each examination item is smaller than the predetermined threshold value.

In addition, the medical assistance device 10 may be configured to determine that the value obtained by averaging the risks for each of the calculated examination items is a risk value relating to the complex disease state. In this case, the medical assistance device 10 may be configured to calculate the value obtained by averaging the risks for each examination item by using an expression of “average value=total value of risks for each examination item÷number of examination items.”

In addition, the medical assistance device 10 may be configured to calculate the risk for each examination item by using an expression of “risk=(examination data of patient−reference value)÷reference value” or an expression of “risk=(reference value−examination data of patient)÷reference value.” Specifically, for the examination item considered as an abnormal value when the examination item exceeds a reference value, a configuration may be adopted to use an expression of “risk=(examination data of patient−reference value)÷reference value,” and for the examination item considered as the abnormal value when the examination item is smaller than the reference value, such as HDL cholesterol, a configuration may be adopted to an expression of “risk=(reference value−examination data of patient)÷reference value.”

In addition, the medical assistance device 10 may be configured to calculate the risk for each examination item by using an expression of “risk=(examination data of patient−reference value)÷reference value×adjustment coefficient.” The adjustment coefficient is a “weight value” indicating an importance degree assigned to the complex disease state by each examination item, and may be determined in advance for each examination item. In addition, the medical assistance device 10 may be configured to add a number obtained by multiplying 0.1 by “the number of items whose examination data exceeds the reference value in the examination items” to the value obtained by averaging the calculated risks for each examination item, and when the value obtained by averaging the risks for each examination item after the addition is equal to or greater than a predetermined threshold value, the medical assistance device 10 may be configured to determine that the patient is in the complex disease state.

FIG. 5 is a view showing an example of a case where the complex disease state is determined based on a determination method 2. Points not particularly mentioned may be the same as those in FIG. 4. Each numerical value in FIG. 5 indicates the risk calculated based on measurement data in FIG. 3. In addition, the risk value of the complex disease state (risk value relating to the complex disease state) in FIG. 5 is obtained by adding the number obtained by multiplying 0.1 by “the number of items whose examination data exceeds the reference value in the examination items” to the value obtained by averaging the risks for each examination item. In the determination method 2, the number obtained by multiplying 0.1 by the number of items whose examination data exceeds the reference value in the examination items is added to the risk value of the complex disease state. Therefore, a magnitude of the number of the examination items exceeding the reference value affects the risk value. In this manner, the risk of the complex disease state can be properly expressed.

When the determination method 2 is used, the medical assistance device 10 may determine that the patient corresponds to the individual disease in which one or more items whose risk is equal to or greater than a predetermined value (for example, 0) in one or more examination items corresponding to the individual disease. For example, in the example in FIG. 5, in a case of the subject F, the risk of chest circumference, BMI, LDL cholesterol, systolic blood pressure, and diastolic blood pressure is equal to or greater than the predetermined value. Therefore, the medical assistance device 10 may determine that the subject F corresponds to the individual diseases such as obesity, dyslipidemia, and hypertension.

Subsequently, the medical assistance device 10 outputs an improvement target to be achieved by the patient to improve the complex disease state, when it is determined that the patient is in the complex disease state (S12). A configuration may be adopted such that the improvement target is displayed on the doctor terminal 30, and is set (determined) by a confirmation (approval) of a doctor. In addition, a configuration may be adopted to receive correction of the output improvement target from the doctor.

The improvement target may be an improvement target value for an examination value included in the personal data of the patient. In addition, the improvement target may be a self-improvement target of the patient himself/herself, or may be an improvement target reviewed between the doctor and the patient. For example, when the complex disease state is the metabolic syndrome, examples of the improvement target include causing the blood pressure to be lower than 135/85 mmHg, causing the LDL cholesterol to be lower than 140 mg/dl, causing the abdominal circumference to be equal to or smaller than 85 cm, and the like.

The medical assistance device 10 may be configured to set an improvement target value for the examination value included in the personal data of the patient, based on a combination of the individual diseases in the complex disease state of the patient or a combination of the individual disease and the personal data. A specific improvement target value may be determined in advance in accordance with the combination of the individual diseases in the complex disease state or the combination of the individual disease and the personal data in the complex disease state.

In addition, a configuration may be adopted such that the improvement target value is set to a more severe value (for example, a value close to a normal value and the same applies to the followings) as the number of the individual diseases included in the combination of the individual diseases in the complex disease state increases. In addition, when the age of the patient exceeds a certain threshold value (for example, 45 years or older for the male, 55 years or older for the female, and the like), a configuration may be adopted such that the improvement target value is set to a more severe value than a value when the age does not exceed the threshold value. In addition, when the patient smokes, the improvement target value may be set to a more severe value than a value of the patient who does not smoke. That is, the improvement target value may be set to the more severe value as the number of the individual diseases forming the complex disease state (number of risk factors) increases.

For example, it is assumed that the improvement target of the LDL cholesterol for the patient suffering from only dyslipidemia is set to be lower than 140 mg/dl. In this case, the medical assistance device 10 may be configured to set the improvement target of the LDL cholesterol for the patient suffering from dyslipidemia and diabetes to be lower than 120 mg/dl which is closer to the normal value and lower than 140 mg/dl. In addition, the medical assistance device 10 may be configured to set the improvement target of the LDL cholesterol for the patient suffering from dyslipidemia and coronary disease to be lower than 100 mg/dl which is closer to the normal value and lower than 140 mg/dl. In addition, the medical assistance device 10 may be configured to set the improvement target of the LDL cholesterol for the patient suffering from dyslipidemia, diabetes, and coronary disease to be lower than 70 mg/dl which is closer to the normal value and lower than 100 mg/dl that is the improvement target of the LDL cholesterol for the patient suffering from dyslipidemia and coronary disease.

In addition, in a case of a disease that is one of lifestyle habit-related diseases, the improvement target may be an improvement target for a lifestyle habit of the patient. For example, the improvement target includes “to have only one dish with a strong taste per meal” in a case of a meal, “to walk after a meal” in a case of activity, “to sleep one hour earlier than usual” in a case of sleeping, or the like.

Subsequently, the medical assistance device 10 receives settings of a behavior target to be handled by the patient to achieve the improvement target from the patient (S13). The behavior target may be classified into a plurality of categories. For example, the plurality of categories may include at least one of “stress,” “sleeping,” “activity,” “meal,” “smoking,” and “alcohol drinking.” The activity includes not only an exercise but also a general body movement such as walking and the like. That is, the behavior target to be handled by the patient to achieve the improvement target may include at least one of a behavior target relating to stress improvement of the patient, a behavior target relating to improvement of sleeping hours of the patient, a behavior target relating exercise amount improvement of the patient, a behavior target relating to the improvement in dietary habits of the patient, a behavior target relating to smoking amount reduction of the patient, and a behavior target relating to alcohol drinking amount reduction of the patient. For example, as the behavior targets, specific behavior targets may be set in accordance with a treatment plan of the patient, such as walking for 30 minutes or longer, not eating the meal after 21:00 at night, always eating breakfast, not alcohol drinking after 20:00, or the like, and the behavior target of each category relating to the lifestyle habit of the above-described patient may be scored. The category is merely an example, and the present embodiment is not limited thereto.

The medical assistance device 10 may be configured to present the patient with options of the behavior target to be handled by the patient to achieve the improvement target, and to receive selection of the behavior target to be handled by the patient himself/herself from the presented options. The medical assistance device 10 may be configured to determine the options of the behavior target to be handled by the patient to achieve the improvement target, based on the personal data of the patient, the improvement target of the patient, and/or the complex disease state or the like of the patient. For example, when the complex disease state of the patient is the metabolic syndrome corresponding to hypertension and diabetes, the improvement target is causing the blood pressure to be lower than 130/80 mmHg, causing a blood glucose control target to be lower than HbA1c 6.0%, and the age of the patient is 60 years or older, as the options of the behavior target, the medical assistance device 10 may be configured to present the behavior targets such as “to build up stamina by walking fast,” “not to eat a meal with high carbohydrate,” and “not to drink alcohol.”Subsequently, the medical assistance device 10 receives an answer to questionnaire relating to the behavior target to be handled by the patient from the patient (S14). More specifically, the questionnaire may be a questionnaire for receiving a confirmation of the patient to perform the behavior target to be handled by the patient. The questionnaire may include a questionnaire to directly confirm a performance result (handling status) of the behavior target selected by the patient in Step S13. For example, the questionnaire may include a questionnaire for directly confirming the performance result of the behavior target selected by the patient, such as a questionnaire of “did you try not to eat a meal with high carbohydrate today?” when the patient selects “not to eat the meal with high carbohydrate” as the behavior target, and a questionnaire of “how many minutes in total did you exercise (including fast walking or housework) to such an extent that you sweat?” when the patient selects “to walk for 30 minutes or longer.” In addition, the medical assistance device 10 receives biological information of the patient from a wearable device worn by the patient.

Subsequently, the medical assistance device 10 outputs a score relating to an achievement degree of the behavior target to be handled by the patient, and an advice relating to the achievement degree of the behavior target to be handled by the patient, based on the answer to questionnaire and/or the biological information received in Step S14 (S15). As described above, since the behavior target may be divided into the plurality of categories, the score may also be output by being divided into the same categories as those of the behavior target. As in the behavior target, for example, the plurality of categories may include at least one of “stress,” “sleeping,” “exercise,” “meal,” “smoking,” and “alcohol drinking.” That is, the score may include at least one of a score relating to stress, a score relating to sleeping, a score relating to exercise, a score relating to meal, a score relating to smoking, or a score relating to alcohol drinking. In addition, the score may be calculated such that the score is a high value when the patient actively takes behavior in achieving the improvement target, and the score is a low value when the patient does not actively take behavior in achieving the improvement target. For example, the scores are exemplified in a form of 80 for sleeping, 59 for exercise, 78 for meal, 100 for smoking/alcohol drinking, and the like. In addition, these scores may be used as the behavior target. In addition, the advice may include a content of praising the patient (for example, “do your best in this condition” or the like) when the patient actively takes behavior in achieving the improvement target, and a content of promoting the improvement (for example, “the disease will worsen if you do not reduce alcohol drinking” or the like) when the patient does not actively takes behavior in achieving the improvement target.

In a process procedure in Step S15, the medical assistance device 10 may visualize and display a current or future image of an appearance and/or a current or future image of an organ of the patient, based on the improvement target, the behavior target, the questionnaire, or the advice.

Examples of the image of the organ which is displayed by the medical assistance device 10 include a brain, a liver, an eye, a kidney, a heart, a blood vessel, a lung, a nerve, and the like. In this manner, a future state based on a current state of the patient, the improvement target, the behavior target, the questionnaire, the advice, or the like is visualized and displayed for each organ. In this manner, the patient himself/herself can visually recognize and understand the current and future states.

In addition, the medical assistance device 10 may provide the patient with specific medical information on the disease or the examination item relating to each organ in a form of “trivia” by using a video and/or writing, or the like. For example, in a case of the liver as the organ, an explanatory video relating to the disease such as fatty liver, hepatitis, liver cirrhosis, liver cancer, and the like may be provided. In addition, in a case of dyslipidemia as the disease, the medical assistance device 10 may display a medical explanatory document or the like relating to the disease, such as “dyslipidemia is a disease in which LDL cholesterol (bad cholesterol) or neutral fat (triglyceride) has an abnormal value, when cholesterol is accumulated on a vascular wall, a plaque (lump) is formed, and a passage of blood is clogged, thereby leading to arteriosclerosis.”

In this way, the patient is enabled to visually understand each disease and the state of the complex disease, and medical information relating thereto. Accordingly, the patient himself/herself can understand his/her own state. As a result, a behavior change in his/her lifestyle habit and the like is promoted, based on an improvement plan or the like.

In addition, the medical assistance device 10 may display data that enables the patient to confirm a transition or a distribution of an input value such as the personal data or the like (for example, personal data such as a body weight, a blood pressure, a blood glucose level which are measured by the patient, and the like) of the patient during a period of one week, one month, or one year. Furthermore, the medical assistance device 10 may display information that enables the patient to confirm a transition of each score, contents of the answer to questionnaire, the advice for each lifestyle habit category (stress, sleeping, activity, meal, smoking, alcohol drinking, and the like) during a period of one day, one week, or one month, and the like. As a result, the patient himself/herself can understand his/her own state, and can further promote the behavior change in his/her own lifestyle habit and the like, based on the improvement plan or the like.

Furthermore, the medical assistance device 10 may be configured to display a quiz or the like to improve literacy relating to a health or a lifestyle habit-related disease. For example, a form of the quiz is not particularly limited, and examples thereof include a form in which one quiz is displayed every day such that the patient answers the quiz. A configuration is devised such that five points are assigned to a correct answer, three points are assigned even to a wrong answer, and the like. In this manner, the patient can learn knowledge or information on the health or the lifestyle habit-related disease while enjoying the learning, and can immediately utilize the learned health knowledge. Therefore, health literacy can be improved, and the behavior change can be promoted.

A configuration may be adopted such that the process procedure in Steps S10 to S12 described above is executed when the patient receives the examination of the doctor, and the process procedure in Steps S13 to S15 is repeatedly executed every day until the next examination from when the examination is completed.

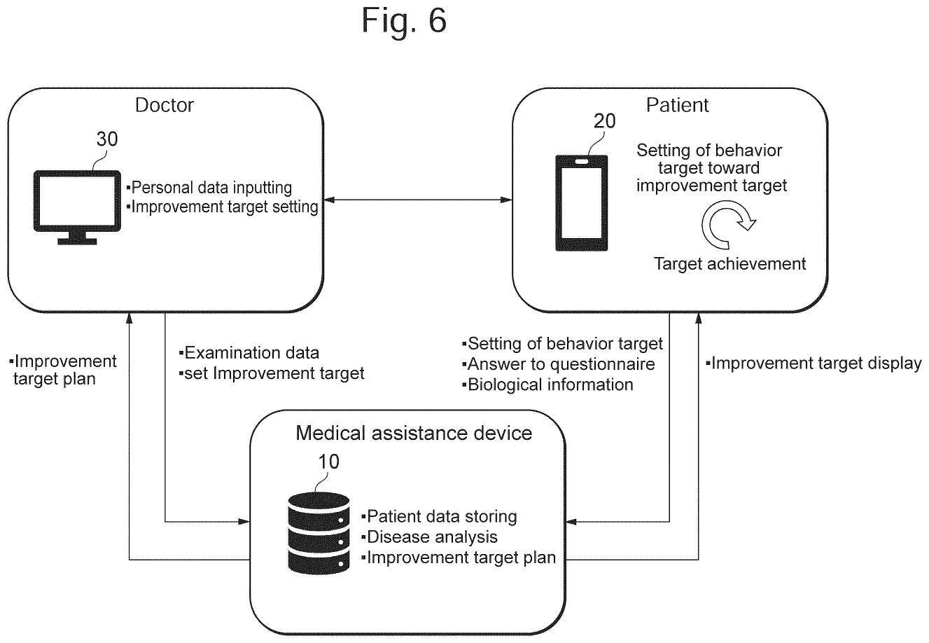

FIG. 6 is a schematic view showing a flow of medical assistance using the medical assistance system 1. As shown in FIG. 6, the doctor examines the patient, and inputs the personal data (various types of examination data and the like) of the patient from the doctor terminal 30. In addition, the patient inputs the personal data (various types of medical interview data) of the patient from the patient terminal 20. In addition, the doctor performs setting or the like of an improvement target to be achieved by the patient, based on the improvement target, the doctor' own medical knowledge, and the like which are output from the medical assistance device 10 and displayed on the doctor terminal 30.

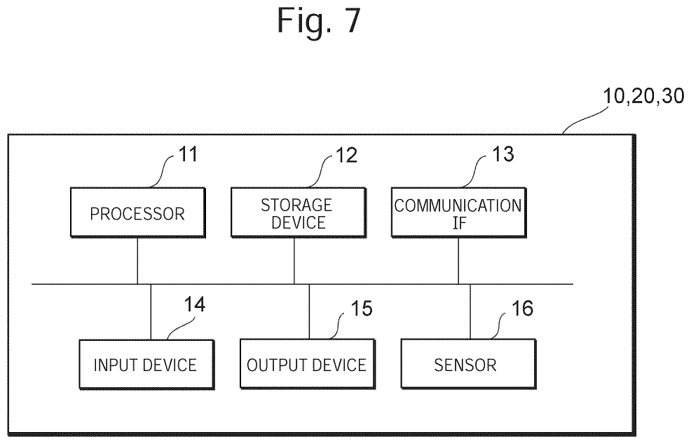

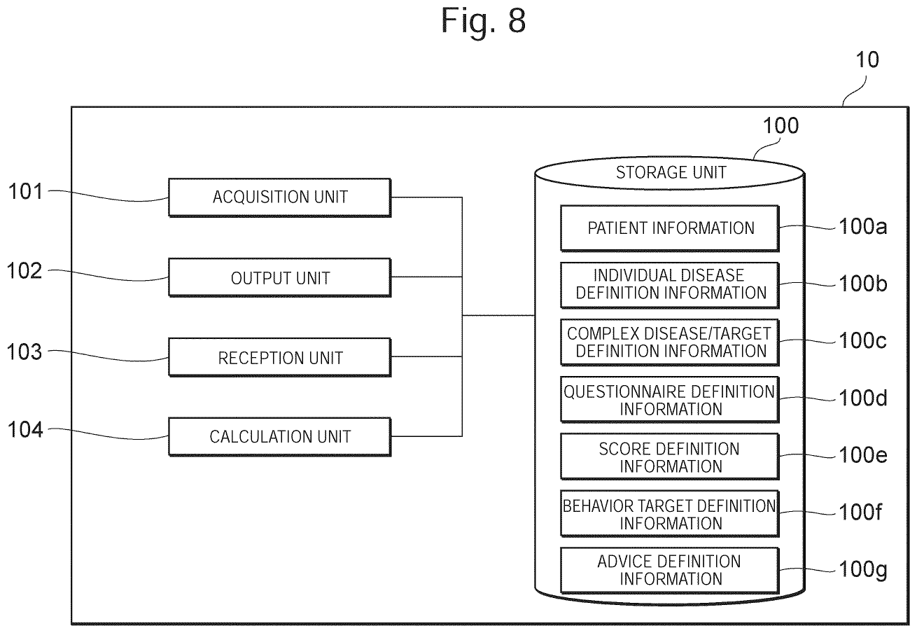

The patient who has completed the examination recognizes his or her own disease state or improvement target by confirming the disease state or the improvement target displayed on the patient terminal 20. In addition, the patient sets a daily behavior target for achieving the improvement target on a screen of the patient terminal 20, and answers a daily questionnaire displayed on the patient terminal 20. When the questionnaire is answered, a score and an advice relating to the achievement degree of the behavior target are displayed on the patient terminal 20, and the patient can look back on the daily behavior. In addition, the patient can daily and continuously review his/her own behavior by repeatedly setting the behavior target, answering to the questionnaire, and confirming the score and the advice, and as a result, a recovery from the disease can be achieved.

<Hardware Configuration> FIG. 7 is a view showing a hardware configuration example of the medical assistance device 10, the patient terminal 20, and the doctor terminal 30. The medical assistance device 10, the patient terminal 20, and the doctor terminal 30 include a processor 11 such as a central processing unit (CPU), a graphical processing unit (GPU), and the like, a storage device 12 such as a memory, a hard disk drive (HDD) and/or a solid state drive (SSD), and the line, a communication interface (IF) 13 for performing wired or wireless communication, an input device 14 that receives an input operation, and an output device 15 that outputs information. For example, the input device 14 is a keyboard, a touch panel, a mouse, and/or a microphone, and the like. For example, the output device 15 is a display, a touch panel, and/or a speaker, and the like. The medical assistance device 10 may be configured to include one or a plurality of physical servers and the like, may be configured by using a virtual server operated on a hypervisor, or may be configured by using a cloud server.

<Functional Block Configuration> (Medical assistance device) FIG. 8 is a view showing a functional block configuration example of the medical assistance device 10. The medical assistance device 10 includes a storage unit 100, an acquisition unit 101, an output unit 102, a reception unit 103, and a calculation unit 104. The storage unit 100 can be realized by using the storage device 12 provided in the medical assistance device 10. In addition, the acquisition unit 101, the output unit 102, the reception unit 103, and the calculation unit 104 can be realized in such a manner that the processor 11 of the medical assistance device 10 executes a program stored in the storage device 12. In addition, the program can be stored in a storage medium. The storage medium in which the program is stored may be a non-transitory computer readable medium. The non-transitory storage medium is not particularly limited and, for example, may be a storage medium such as a USB memory, a CD-ROM, or the like.

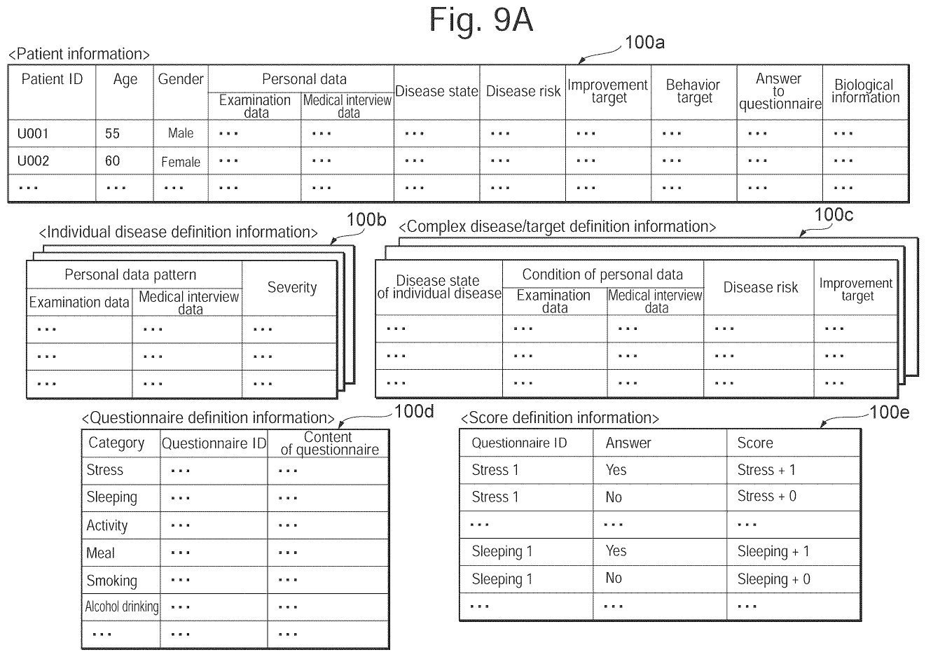

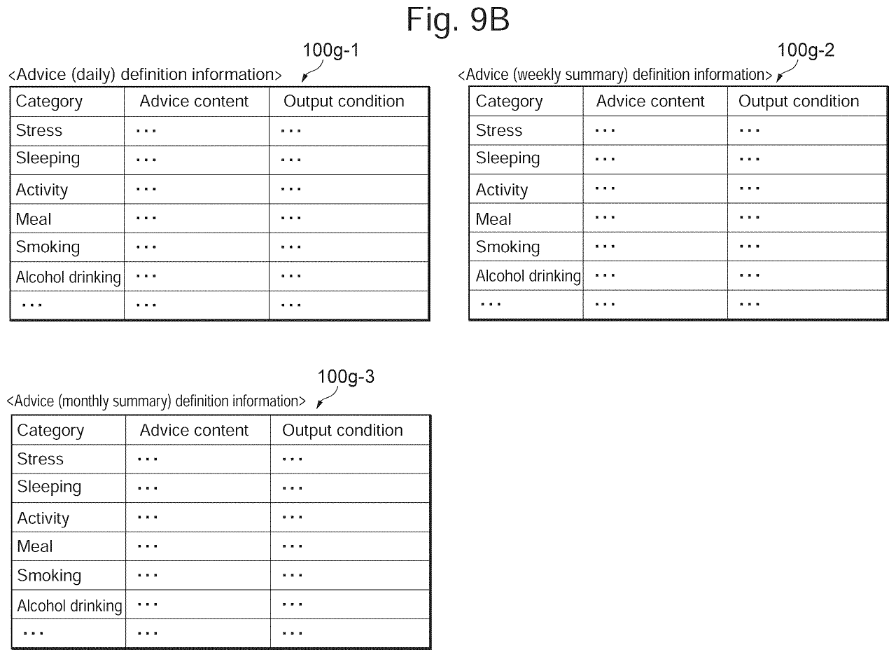

The storage unit 100 stores patient information 100a that stores various types of information relating to the patient, individual disease definition information 100b that defines a relationship between the personal data and the disease state of the individual disease, complex disease/target definition information 100c that defines the improvement target when a combination of the disease states of individual diseases or a combination of the disease state of the individual disease and a personal data condition is the complex disease state, questionnaire definition information 100d that defines the content of the questionnaire relating to the behavior target to be handled by the user, score definition information 100e that defines a method for calculating the score from the answer to questionnaire, behavior target definition information 100f that defines the behavior target to be presented to the patient as the option, and advice definition information 100g that defines the content of the advice to be presented to the patient. The complex disease/target definition information 100c may be referred to as “target definition information.”FIG. 9A is a view showing an example of the patient information 100a, the individual disease definition information 100b, the complex disease/target definition information 100c, the questionnaire definition information 100d, and the score definition information 100e. First, the patient information 100a will be described. A “patient identification (ID)” is an identifier for identifying the patient. “Age” indicates the age of the patient. “Gender” indicates the gender of the patient. Various types of examination data and various types of medical interview data of the patient are stored in the “personal data.” The “disease state,” the “disease risk,” and the “improvement target” respectively store the disease state (disease state of each individual disease and complex disease state), the disease risk, and the improvement target which are output from the medical assistance device 10, based on the personal data. The “behavior target” stores the behavior target selected by the patient. The “answer to questionnaire” stores the content of the answer to questionnaire answered by the patient. The “biological information” stores information such as daily activity amount (number of steps, a walking distance, consumed calories, and the like), sleeping information, heart rates obtained from the wearable device, or the like.

Next, the individual disease definition information 100b will be described. The individual disease definition information 100b shown in FIG. 9A is defined for each individual disease. An “personal data pattern” stores a pattern of the examination data and the medical interview data corresponding to the individual disease (that is, a predetermined reference corresponding to the individual disease). The “severity” corresponds to the disease state of the individual disease, and indicates the severity of the individual disease corresponding to the personal data pattern. A configuration may be adopted such that the severity is expressed in a plurality of levels (stages), and a higher level indicates a more severe disease. For example, in the individual disease definition information 100b corresponding to the hypertension, a configuration may be adopted such that the personal data pattern is defined as hypertension in a level 5 when the systolic blood pressure is 180 mmHg or higher and the diastolic blood pressure is 110 mmHg or higher, and as hypertension in a level 4 when the systolic blood pressure is 160 to 179 mmHg and the diastolic blood pressure is 100 to 109 mmHg. In addition, the personal data pattern which is not included in the individual disease definition information 100b means that the patient does not correspond to the individual disease. For example, when the personal data pattern in which the systolic blood pressure is 130 mmHg or lower and the diastolic blood pressure is 80 mmHg or lower is not defined in the individual disease definition information 100b corresponding to hypertension, the medical assistance device 10 can determine that the patient having the systolic blood pressure of 130 mmHg or lower and the diastolic blood pressure of 80 mmHg or lower does not correspond to hypertension.

Next, the complex disease/target definition information 100c will be described. The complex disease/target definition information 100c shown in FIG. 9A is defined for each complex disease state. For example, when the medical assistance device 10 corresponds to determination of a complex disease state A and a complex disease state B, the storage unit 100 stores the complex disease/target definition information 100c corresponding to the complex disease state A and the complex disease/target definition information 100c corresponding to the complex disease state B. The “disease state of the individual disease” and the “condition of the personal data” store a combination of the disease states of the individual diseases corresponding to the complex disease state, or a combination of the disease state of the individual disease corresponding to the complex disease state and the condition of the personal data. When the complex disease state corresponds to the combination of the disease states of the individual diseases, the “condition of the personal data” may be omitted. Although the patient does not currently suffers from the disease, the “disease risk” stores information indicating a disease name of the disease from which the patient possibly suffers and a degree of the possibility that the patient suffers from the disease, which are presumed from the combination of the disease states of the individual diseases of the patient or the combination of the disease state of the individual disease of the patient and the personal data condition. Here, the patient with the metabolic syndrome has a possibility of suffering from a cerebrocardiovascular disease or a coronary disease in the future, and it is known that the risk of suffering from the cerebrocardiovascular disease or the coronary disease is higher as each individual disease forming the metabolic syndrome is more severe. Therefore, the “disease risk” of the complex disease/target definition information 100c corresponding to the metabolic syndrome may store a numerical value indicating the risk of suffering from the cerebrocardiovascular disease or the coronary disease (for example, as the value is greater, the possibility of suffering from the disease in the future is higher) or information relating to a color indicating the severity (magnitude of the risk). The “improvement target” stores the improvement target of the numerical value to be achieved by the patient to improve the complex disease state.

When the complex disease state is determined in accordance with the determination method 1, the patient can correspond to the complex disease state by using the individual disease definition information 100b and the complex disease/target definition information 100c which are shown in FIG. 9A. On the other hand, when the complex disease state is determined in accordance with the determination method 2, instead of the items shown in FIG. 9A, the complex disease/target definition information 100c may store data indicating a processing logic required to execute the determination method 2 and the reference value of each examination item as common data for each complex disease state, and data in which the “disease risk” and the “improvement target” are associated with each other as data for each complex disease state. In addition, instead of the items shown in FIG. 9A, as data for each individual disease, the individual disease definition information 100b may store a “predetermined value” used to determine whether or not the patient corresponds to the individual disease, and data indicating a correspondence relationship between the calculated risk value and the severity for each examination item relating to the individual disease.

Next, the questionnaire definition information 100d will be described. The “category” indicates a category of the behavior target to be handled by the user. In the example in FIG. 9A, examples of the category example include stress, sleeping, exercise, meal, smoking, and alcohol drinking, but the present disclosure is not limited thereto. A “questionnaire ID” indicates an identifier for identifying the questionnaire. The “content of the questionnaire” is stored in the “content of the questionnaire.”

Next, the score definition information 100e will be described. A “questionnaire ID” indicates an identifier for identifying the questionnaire. The questionnaire ID corresponds to the questionnaire ID of the questionnaire definition information 100d. The “answer” indicates the answer to questionnaire. The “score” indicates a value added to the score. In the example in FIG. 9A, when the patient answers “yes” to the questionnaire of a stress 1, a case where one is added to the score of the category of the stress is shown. Similarly, when the patient answers “no” to the questionnaire of the stress 1, a case where nothing is added to the score of the category of the stress is shown. The score definition information 100e shown in FIG. 9A is an example, and the present disclosure is not limited thereto. For example, the score definition information 100e may store a logic for calculating the score by combining one or a plurality of answers to the questionnaire. For example, when the answer to questionnaire 1 belonging to the stress is “yes,” the answer to questionnaire 5 belonging to the stress is “yes,” and the answer to questionnaire 1 belonging to the sleep is “no,” a logic in which three are added to the score of the category of the stress may be stored. In addition, the score definition information 100e may be further defined such that the biological information and information relating to the score to be added are associated with each other. For example, information such as adding one to the score of the activity when a walking distance is equal to or greater than a predetermined threshold value, adding one to the score of the sleeping when sleeping hours are equal to or greater than a predetermined time, or the like may be defined.

FIG. 9B is a view showing an example of the advice definition information 100g. As shown in FIG. 9B, the advice definition information 100g may be divided into advice (daily) definition information 100g-1 that defines the content of a short-term advice (for example, a daily review) for the behavior content to be handled by the patient, advice (weekly summary) definition information 100g-2 that defines the content of a medium-term advice (for example, a weekly review) for the behavior content to be handled by the patient, and advice (monthly summary) definition information 100g-3 that defines the content of a long-term advice (for example, a monthly review) for the behavior content to be handled by the patient. The “category” indicates the category of the advice. The stored category may be the same as the category of the behavior target. The “advice content” stores a phrase of the advice to be output. One advice content may be stored for each category, or a plurality of advice contents may be stored. The “output condition” stores a condition for determining whether or not to output any advice. The output condition may be defined for each category, or may be defined for each advice content. Returning to FIG. 8, the description will be continued.

The acquisition unit 101 acquires the personal data including the examination data and the medical interview data of the patient from the patient terminal 20 or the medical assistance device 10. The acquisition unit 101 may be configured to acquire the personal data acquired by the wearable device worn by the patient.

The output unit 102 determines the disease state of the patient for the individual disease forming the complex disease, based on the personal data of the patient, and outputs the improvement target to be achieved by the patient to improve the complex disease state when the combination of the disease states of the individual diseases or the combination of the disease state of the individual disease and the personal data is the complex disease state. The improvement target may include an improvement target value for the examination value included in the personal data of the patient. In addition, the personal data of the patient includes data relating to the lifestyle habit of the patient, and the improvement target may include the improvement target for the lifestyle habit of the patient. The lifestyle habit may include at least one of the stress of the patient, the sleeping hours of the patient, the exercise amount of the patient, the dietary habit of the patient, the smoking amount of the patient, and the alcohol drinking amount of the patient.

In addition, the output unit 102 may be configured to set the improvement target for the examination value included in the personal data of the patient, based on the combination of the disease states of the individual diseases or the combination of the disease state of the individual disease and the personal data.

In addition, the output unit 102 may be configured to determine the disease state of the patient by referring to the individual disease definition information 100b, to acquire the improvement target by referring to the complex disease/target definition information 100c when the combination of the disease states of the individual diseases or the combination of the disease state of the individual disease and the personal data is the complex disease state, and to output the acquired improvement target as the improvement target to be achieved by the patient. For example, when the personal data of the patient corresponds to any of the records of the “personal data pattern” defined by the individual disease definition information 100b corresponding to the individual disease A, the output unit 102 may be configured to determine that the patient corresponds to the individual disease A, and may determine the disease state (severity) of the individual disease A from which the patient suffers by referring to a “severity” field of the corresponding record. In addition, the output unit 102 may repeat the same process for each individual disease. In this manner, the output unit 102 may be configured to determine whether the patient corresponds to any individual disease in the respective individual diseases and the disease state (severity) of the corresponding individual disease. Subsequently, the output unit 102 may refer to the complex disease/target definition information 100c corresponding to the complex disease state A. When the disease state of each individual disease of the patient and the personal data of the patient correspond to any of the records of the “individual disease state” and the “condition of personal data” which are defined by the complex disease/target definition information 100c, the output unit 102 may be configured to determine that the patient is in the complex disease state A, and to acquire the improvement target of the patient by referring to the “improvement target” field of the corresponding record. In addition, the output unit 102 may repeat the same process for each complex disease state. In this manner, the patient may be configured to determine whether the patient corresponds to any complex disease state in the respective complex disease states, and to acquire the improvement target when the patient corresponds to the complex disease state.

In addition, with regard to the individual disease forming the complex disease, the output unit 102 may be configured to calculate the risk for each examination item by using the reference value of one or more examination items relating to the individual disease, and the examination data for the examination item obtained by examining the patient, and to determine that the patient is in the complex disease state, when the calculated risk for each examination item satisfies a predetermined condition. In addition, the output unit may be configured to output the improvement target to be achieved by the patient to improve the complex disease state, when it is determined that the patient is in the complex disease state.

In addition, when the calculated risk for each examination item satisfies the predetermined condition, a value obtained by averaging the calculated risks for each examination item may be equal to or greater than a predetermined threshold value. In this case, the output unit 102 may be configured to output the value obtained by averaging the calculated risks for each examination item, as a value indicating the risk relating to the complex disease state.

In addition, the output unit 102 may be configured to output the improvement target of the patient which is obtained by inputting the personal data of the patient to a trained model having ability to output the improvement target for improving the complex disease state, as the improvement target to be achieved by the patient.

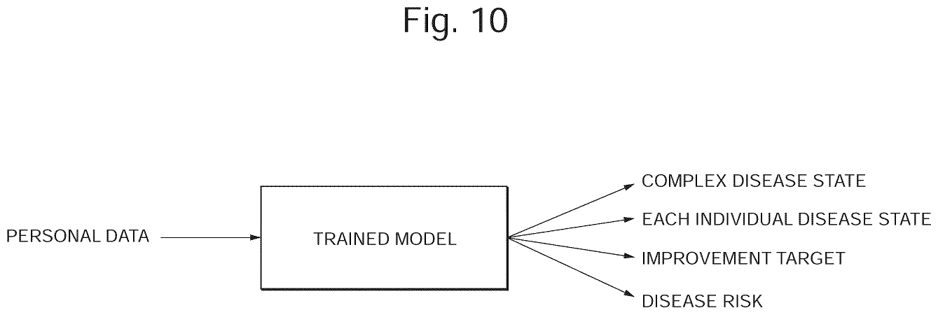

FIG. 10 is a view showing an example of the trained model. The trained model shown in FIG. 10 is a model trained to output a flag indicating whether or not the patient corresponds to the complex disease state, an identifier indicating the disease name of the corresponding complex disease state, a state of each individual disease forming the complex disease state (for example, a flag indicating the presence or absence of the individual disease, a value indicating the severity of the individual disease, or the like), the improvement target, and the disease risk, when the personal data is input. The trained model may be operated to identify whether or not the combination of the personal data corresponds to each individual disease forming the complex disease from the input personal data, and to output the improvement target or the like when the patient corresponds to each individual disease forming the complex disease (when the patient is in the complex disease state). The trained model may be further trained to output the risk value relating to the complex disease state.

In addition, this trained model can be generated in such a manner that a model is trained by generating a large number of training data in which the personal data is used as input data from a large number of past clinical data, and a flag indicating whether or not the patient corresponds to the complex disease state, an identifier indicating the disease name of the corresponding complex disease state, a flag indicating whether or not the patient corresponds to each individual disease forming the complex disease state, a value indicating the severity of the individual disease, the improvement target value, and the disease risk are used as correct answer data. In addition, when the risk value relating to the complex disease state is output from the trained model, the patient can correspond to the complex disease state by causing the risk value relating to the complex disease state to be included in the correct answer data of the teacher data. Although the model may be any algorithm, for example, a neural network, a decision tree, a random forest, a gradient boosting decision tree, or the like can be used. In addition, the trained model may be prepared as a model different for each complex disease state. Returning to FIG. 8, the description will be continued.

In addition, the output unit 102 outputs various types of data to the patient terminal 20 and the doctor terminal 30. The output unit 102 that outputs the data to the patient terminal 20 may be referred to as a first output unit. In addition, the output unit 102 that outputs the data to the doctor terminal 30 may be referred to as a second output unit.

In addition, the output unit 102 (first output unit) may be configured to output the improvement target or the improvement target corrected by the doctor to the patient terminal 20.

The reception unit 103 receives various types of input from the patient terminal 20 or the doctor terminal 30. The reception unit that receives various types of input from the patient terminal 20 (or the patient) may be referred to as a first reception unit. In addition, the reception unit that receives various types of input from the doctor terminal 30 (or the doctor) may be referred to as a second reception unit. The reception unit 103 (first reception unit) receives the behavior target to be handled by the patient to achieve the improvement target from the patient. The reception unit 103 that receives the behavior target from the patient may be referred to as the first reception unit. In addition, the reception unit 103 (first reception unit) may be configured to receive the behavior target to be handled by the patient from the patient, in a plurality of options relating to the behavior target for achieving the improvement target of the patient. In addition, the reception unit 103 (first reception unit) may be configured to refer to the behavior target definition information 100f, and to determine the plurality of options to be presented to the patient by extracting the option corresponding to the personal data of the patient, the improvement target, or the complex disease state of the patient. The output unit 102 may be configured to output the behavior target to be handled by the patient, which is received by the reception unit 103 (first reception unit), to the patient terminal 20 used by the patient and/or the doctor terminal 30 used by the doctor who examines the patient.

In addition, the reception unit 103 (first reception unit) receives an input of the answer to questionnaire relating to the behavior target to be handled by the patient.

In addition, the reception unit 103 (second reception unit) may receive the corrected improvement target from the doctor terminal 30, when the doctor corrects the improvement target.

The calculation unit 104 calculates the score relating to the achievement degree of the behavior target to be handled by the patient, based on the answer to questionnaire received by the reception unit 103 (first reception unit). The calculation unit 104 calculates the score in accordance with a calculation method for the score defined by the score definition information 100e by referring to the score definition information 100e.

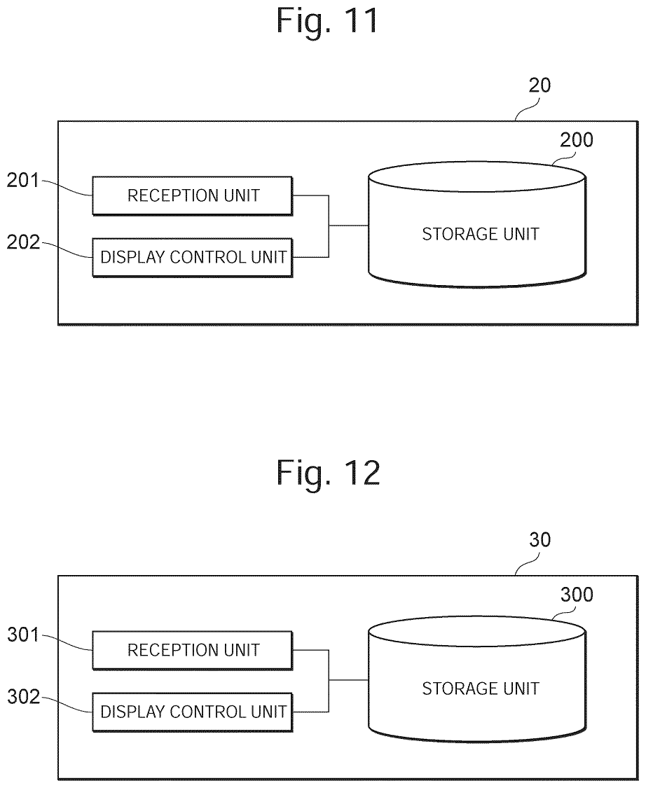

(Patient Terminal) FIG. 11 is a view showing a functional block configuration example of the patient terminal 20. The patient terminal 20 includes a storage unit 200, a reception unit 201, and a display control unit 202. The storage unit 100 can be realized by using the storage device 12 provided in the medical assistance device 10. In addition, the acquisition unit 101, the output unit 102, the reception unit 103, and the calculation unit 104 can be realized in such a manner that the processor 11 of the medical assistance device 10 executes a program stored in the storage device 12. In addition, the program can be stored in a storage medium. The storage medium in which the program is stored may be a non-transitory computer readable medium. The non-transitory storage medium is not particularly limited and, for example, may be a storage medium such as a USB memory, a CD-ROM, or the like.

The storage unit 200 stores various types of data acquired from the medical assistance device 10.

The reception unit 201 receives various types of input detected by the input device 14 provided in the patient terminal 20. For example, the reception unit 201 receives various inputs detected by a touch panel provided in the patient terminal 20.

The display control unit 202 displays data or the like acquired from the medical assistance device 10 on the display provided in the patient terminal 20.

The Web browser installed in the patient terminal 20 may be configured to realize the reception of the information display and the input on the patient terminal 20. In this case, the storage unit 200, the reception unit 201, and the display control unit 202 may be realized by the Web browser installed in the patient terminal 20. Alternatively, a configuration may be adopted such that a dedicated application for realizing a function according to the present embodiment, which is installed in the patient terminal 20, realizes the information display and the input reception on the patient terminal 20. In this case, the storage unit 200, the reception unit 201, and the display control unit 202 may be realized by the dedicated application installed in the patient terminal 20.

(Doctor Terminal) FIG. 12 is a view showing a functional block configuration example of the doctor terminal 30. The doctor terminal 30 includes the storage unit 200, the reception unit 201, and the display control unit 202. The storage unit 200 can be realized by using the storage device 12 provided in the doctor terminal 30. In addition, the reception unit 201 and the display control unit 202 can be realized in such a manner that the processor 11 of the doctor terminal 30 executes a program stored in the storage device 12. In addition, the program can be stored in a storage medium. The storage medium in which the program is stored may be a non-transitory computer readable medium. The non-transitory storage medium is not particularly limited and, for example, may be a storage medium such as a USB memory, a CD-ROM, or the like.

The storage unit 300 stores various types of data acquired from the medical assistance device 10.

The reception unit 301 receives various inputs detected by the input device 14 provided in the doctor terminal 30. For example, the reception unit 301 receives various inputs detected by a keyboard or a mouse provided in the doctor terminal 30. In addition, the reception unit 301 transmits the received various types of input data to the medical assistance device 10.

The display control unit 202 acquires various types of data from the medical assistance device 10, and displays the acquired data to a display provided in the doctor terminal 30.

A configuration may be adopted such that the Web browser installed in the doctor terminal 30 realizes the information display and the input reception on the doctor terminal 30. In this case, the storage unit 300, the reception unit 301, and the display control unit 302 may be realized by the Web browser installed in the doctor terminal 30. Alternatively, a configuration may be adopted such that a dedicated application for realizing a function according to the present embodiment, which is installed in the doctor terminal 30, realizes the information display and the input reception on the doctor terminal 30. In this case, the storage unit 300, the reception unit 301, and the display control unit 302 may be realized by the dedicated application installed in the doctor terminal 30.

<Process Procedure> Subsequently, a process procedure executed by the medical assistance system 1 will be specifically described. In the following description, an example in which the complex disease state is the metabolic syndrome will be described. In addition, an example in which the medical assistance device 10 determines the complex disease state in accordance with the above-described determination method 1 will be described.

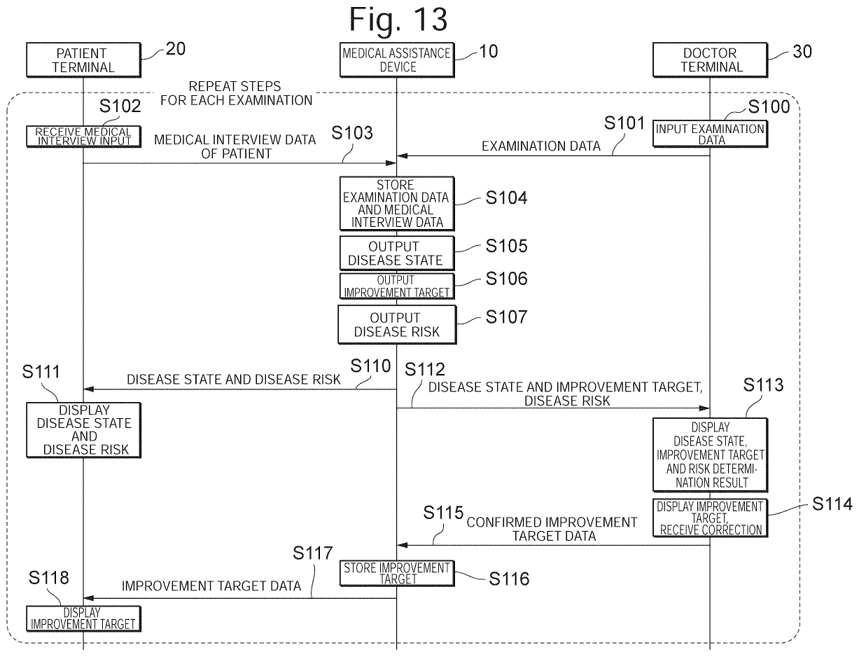

FIG. 13 is a sequence diagram showing an example of a process procedure executed by the medical assistance system 1. Although it is assumed that the process procedure shown in FIG. 13 is repeatedly executed each time the doctor examines the patient, a part of the medical interview (medical interview having the same content for each examination or the like) may be omitted in the second and subsequent examinations.

The medical assistance device 10 receives an input of the examination data of the patient from the doctor, and transmits the input to the doctor terminal 30 (S100 and S101). In addition, the patient terminal 20 receives an input of medical interview data from the patient, and transmits the medical interview data to the medical assistance device 10 (S102 and S103). For example, the doctor may be configured to promote the patient to input the medical interview data from the patient terminal 20 when examining the patient. In addition, the medical interview data may be input to the doctor terminal 30, and may be transmitted to the medical assistance device 10. Subsequently, the medical assistance device 10 stores the examination data and the medical interview data which are received from the patient terminal 20 and the doctor terminal 30, in the patient information 100a (S104).

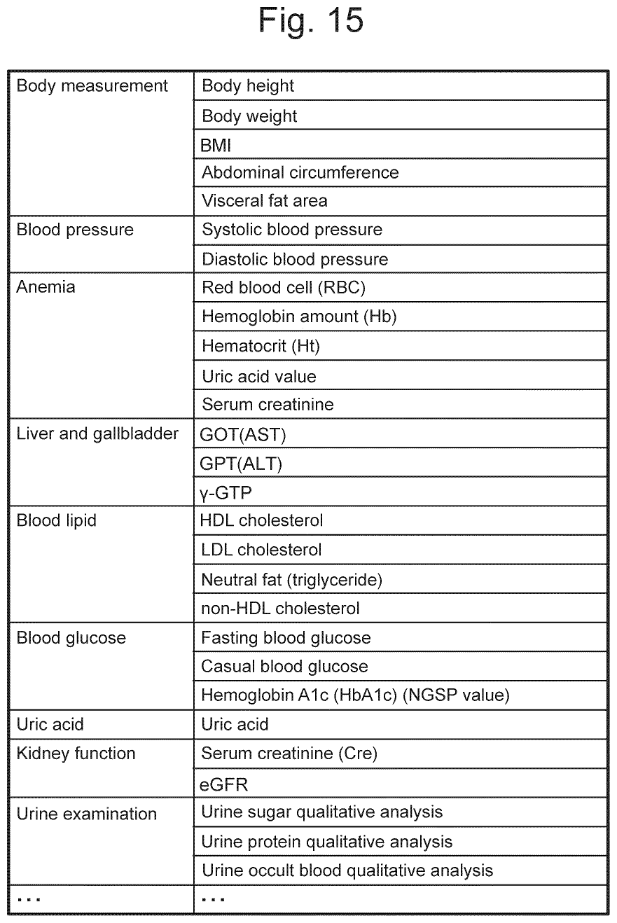

FIG. 15 is a view showing a specific example of the examination data. The examination data shown in FIG. 15 includes data obtained by measuring the body of the patient, such as the body height, the body weight, the abdominal circumference, and the like (body measurement data), data relating to the blood pressure, data relating to anemia, data relating to functions of a liver and a gallbladder, data relating to blood lipids, data relating to a blood glucose level, data relating to uric acid, data relating to a function of a kidney, data obtained by performing urine examination, and the like.

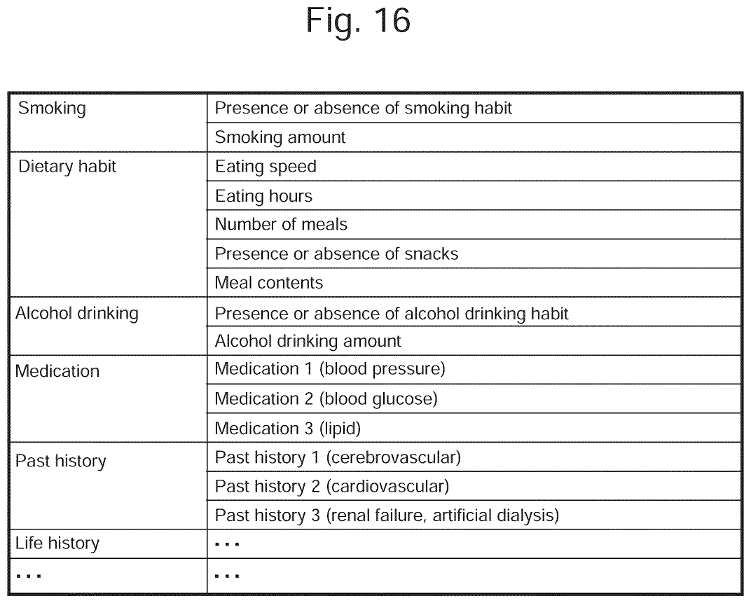

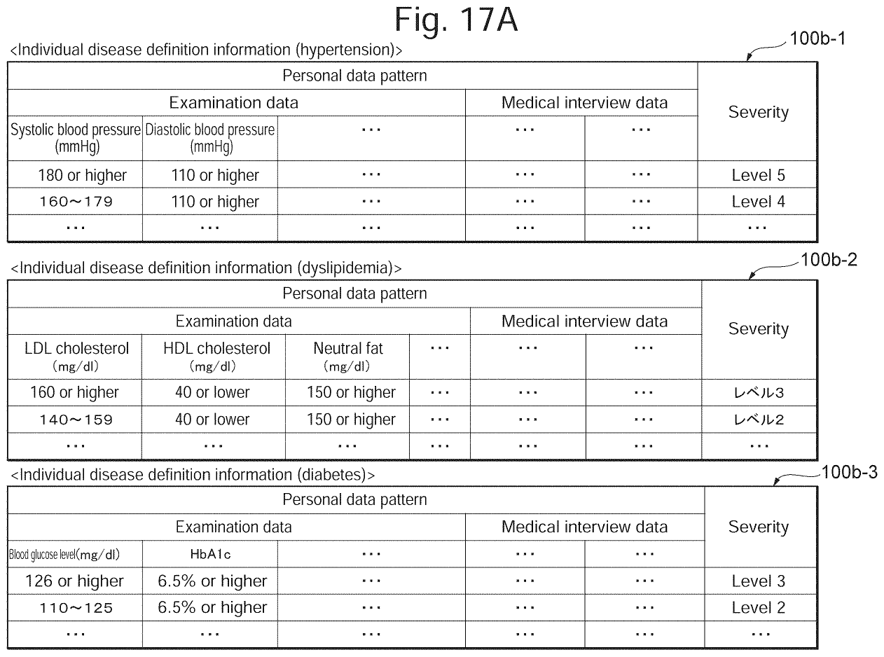

FIG. 16 is a view showing a specific example of the medical interview data. The medical interview data shown in FIG. 16 includes a medical interview relating to smoking, a medical interview relating to a dietary habit, a medical interview relating to alcohol drinking, a medical interview relating to a currently taken drug, a medical interview relating to a past history, a medical interview relating to a life history, and the like. The medical interview data may further include a medical interview required for calculating a score relating to a lifestyle habit of the patient. The score relating to the lifestyle habit may be divided into a plurality of categories. For example, the plurality of categories may be categories of stress, sleeping, exercise, meal, smoking, and alcohol drinking.

Subsequently, the output unit 102 of the medical assistance device 10 refers to the individual disease definition information 100b and the complex disease/target definition information 100c, or uses the trained model described with reference to FIG. 10 to output the disease state, the improvement target, and the disease risk of the patient, and stores the output information in the patient information 100a (S105, S106 and S107). More specifically, the disease state of the patient may be a flag (or a name of the complex disease state) indicating whether or not the patient corresponds to the complex disease state, or a state of each individual disease forming the complex disease state when the patient is in the complex disease state (for example, a flag indicating the presence or absence of the individual disease, a value indicating the severity of the individual disease, and the like). In addition, the improvement target output by the output unit 102 of the medical assistance device 10 may include an improvement target value of the score relating to the lifestyle habit. In addition, the output unit 102 of the medical assistance device 10 may be configured to output a risk value relating to the complex disease state when the patient corresponds to the complex disease state.

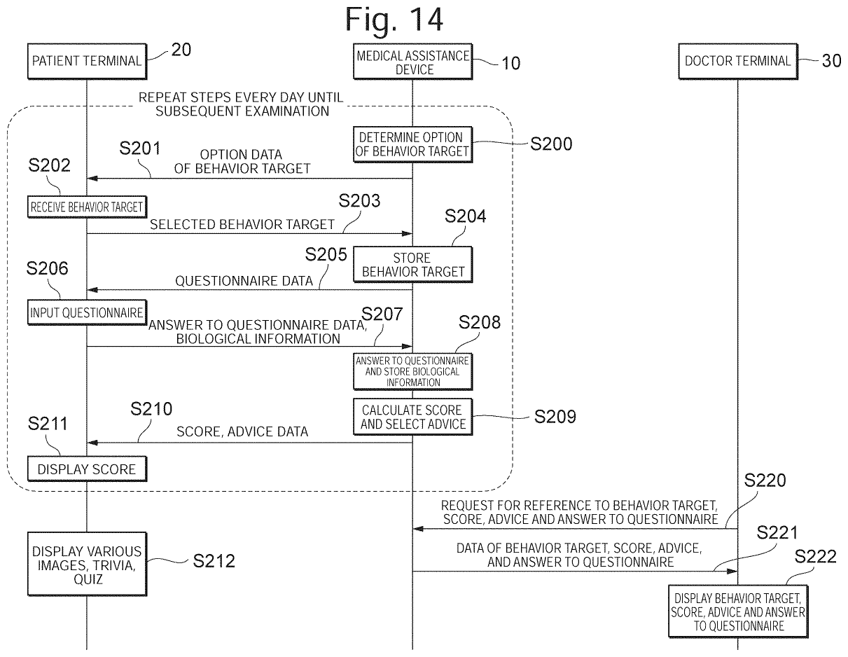

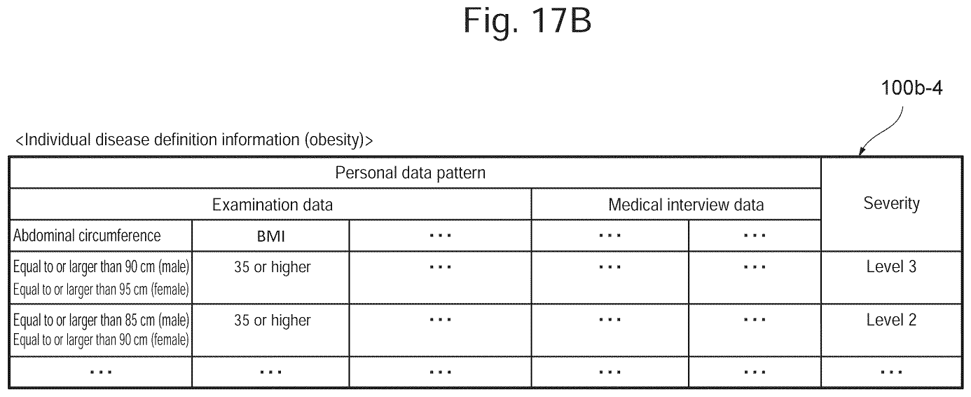

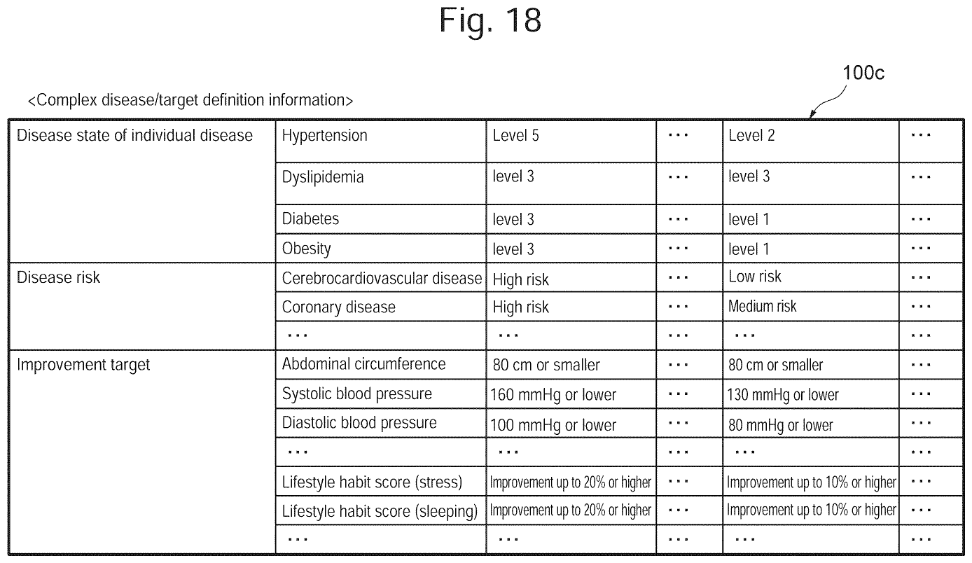

FIG. 14 is a view showing a specific example of the individual disease definition information 100b. FIG. 17A shows individual disease definition information 100b-1 corresponding to hypertension, individual disease definition information 100b-2 corresponding to dyslipidemia, and individual disease definition information 100b-3 corresponding to diabetes. FIG. 17B shows an example of individual disease definition information 100b-4 corresponding to obesity. In addition, FIG. 18 is a view showing a specific example of the complex disease/target definition information 100c. First, the output unit 102 detects a record corresponding to the examination data and the medical interview data of the patient by comparing the “personal data pattern” of the individual disease definition information 100b corresponding to hypertension and the examination data and the medical interview data of the patient with each other. For example, when the examination data of the patient is the systolic blood pressure=190 mmHg and the diastolic blood pressure=120 mmHg, the output unit 102 detects that the examination data and the medical interview data of the patient correspond to the record in a second line. Subsequently, the output unit 102 acquires the severity of the hypertension by referring to the “severity” of the record in the second line. In this manner, the output unit 102 can determine that the patient corresponds to the hypertension and the severity is a level 5. Subsequently, the output unit 102 determines whether or not the patient corresponds to hypertension, dyslipidemia, diabetes, and obesity, and the severity in the corresponding case, by referring to the individual disease definition information 100b-2 corresponding to dyslipidemia, the individual disease definition information 100b-3 corresponding to diabetes, and the individual disease definition information 100b-4 corresponding to obesity. Here, it is determined that the patient corresponds to hypertension (level 5), dyslipidemia (level 3), diabetes (level 3), and obesity (level 3).

Subsequently, the output unit 102 detects the record corresponding to the disease state of the individual disease and the personal data of the patient by comparing the “disease state of the individual disease” and the “personal data condition” of the complex disease/target definition information 100c corresponding to the metabolic syndrome with the individual disease state and the personal data of the patient. As described above, since the disease state of the individual disease of the patient is hypertension (level 5), dyslipidemia (level 3), diabetes (level 3), and obesity (level 3), the output unit 102 detects that the record corresponding to the disease state of the individual disease and the personal data of the patient corresponds to a record in a third column. Subsequently, the output unit 102 acquires the disease risk and the improvement target of the patient by referring to the “disease risk” and the “improvement target” of the record in the third column.

Subsequently, as the disease state of the patient, the output unit 102 outputs a flag indicating that the patient corresponds to the complex disease state (metabolic syndrome), a flag indicating that the patient corresponds to hypertension, a flag indicating that the patient corresponds to dyslipidemia, a flag indicating that the patient corresponds to diabetes, a flag indicating that the patient corresponds to obesity, data indicating that the severity of hypertension is the level 3, data indicating that the severity of the dyslipidemia is the level 3, data indicating that the severity of the diabetes is the level 3, and data indicating that the severity of the obesity is the level 3. In addition, the improvement target, the output unit 102 outputs setting the abdominal circumference to be equal to or smaller than 80 cm, setting the systolic blood pressure to be equal to or lower than 160 mmHg, setting the diastolic blood pressure to be equal to or lower than 100 mmHg, improving the lifestyle habit score relating to stress by 20% or higher, and improving the lifestyle score relating to sleeping by 20% or higher. In addition, as the disease risk, the output unit 102 outputs that the risk of the cerebrocardiovascular disease is high, that the risk of the coronary disease is high, and the like. The disease risk may be expressed by a numerical value. When the output unit 102 outputs the improvement target, the output unit 102 may output a specific numerical value as the lifestyle habit score. For example, the output unit 102 may calculate the lifestyle habit score, based on the medical interview data, and may output a specific target value (for example, score 50×120%=score 60) by multiplying the calculated lifestyle habit score (for example, sleeping=50) by a target value indicated by the improvement target (for example, improving the lifestyle habit score relating to sleeping by 20% or higher). In addition, as the disease state of the patient, the output unit 102 may be configured to further output the number of the individual diseases corresponding to the patient as a risk value relating to the complex disease state (metabolic syndrome). Here, the output unit 102 may be configured to output “four” as the risk relating to the complex disease state. Returning to FIG. 13, the description will be continued.

Subsequently, the output unit 102 transmits the disease state and the disease risk which are output in Step S105 and Step S107 to the patient terminal 20 (S110). The patient terminal 20 displays the disease state and the disease risk which are received from the medical assistance device 10, on the screen (S111).

In addition, the output unit 102 transmits the disease state, the improvement target, and the disease risk which are output in Steps S105 to S107 to the doctor terminal 30 (S112). The medical assistance device 10 displays the disease state, the improvement target, and the disease risk which are received from the medical assistance device 10, on the screen (S113). When the doctor wants to correct the improvement target, the doctor terminal 30 may be configured to receive the correction of the improvement target (S114). In this manner, when examining the patient, the doctor can present the improvement target output from the medical assistance device 10 to the patient, and can correct the improvement target in consultation with the patient.

When the correction of the improvement target is received from the doctor, the doctor terminal 30 transmits the corrected improvement target to the medical assistance device 10 (S115). The medical assistance device 10 updates the “improvement target” of the patient information 100a with the corrected improvement target (S116). Subsequently, the output unit 102 of the medical assistance device 10 transmits the improvement target data stored in the “improvement target” of the patient information 100a to the patient terminal 20 (S117). The patient terminal 20 displays the improvement target received from the medical assistance device 10 on the screen (S118).

FIG. 14 is a sequence diagram showing an example of a process procedure executed by the medical assistance system 1. Although it is assumed that the process procedure shown in FIG. 14 is repeatedly executed every day until the subsequent examination, the present disclosure is not necessarily limited thereto. For example, the process may be repeatedly executed every other day, or may be repeatedly executed every week.

The output unit 102 of the medical assistance device 10 determines the option of the behavior target to be handled by the patient to achieve the improvement target, based on the personal data of the patient, the improvement target of the patient, and/or the complex disease state of the patient (S200). As the option of the behavior target to be presented to the patient, the output unit 102 may be configured to determine the option of the behavior target that satisfies a selection reference by comparing the personal data of the patient, the improvement target of the patient, and/or the complex disease state of the patient with the selection reference defined in the behavior target definition information 100f.

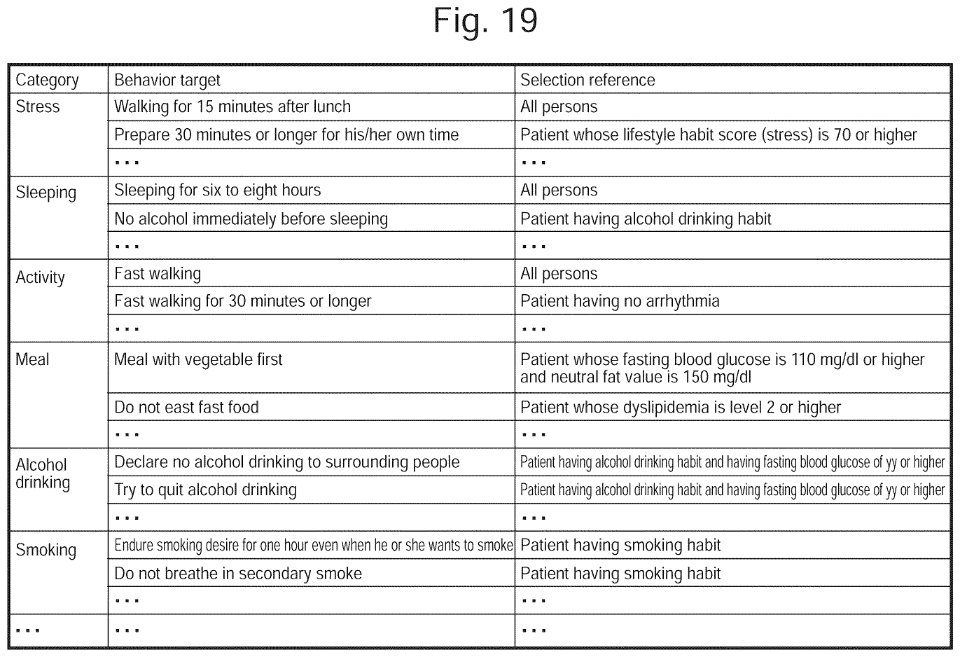

FIG. 19 is a view showing an example of the behavior target definition information 100f. The “category” indicates a category of the behavior target. The “behavior target” indicates specific content of the behavior target. The “selection reference” indicates a reference of the patient for whom the behavior target is selected. For example, according to FIG. 19, the behavior target of “eat meals starting from vegetables” means that the behavior target is extracted as the option when the fasting blood glucose of the examination data is 110 mg/dl or higher and the neutral fat value is 150 mg/dl. Although not shown in FIG. 19, the selection reference such as the “patient whose complex disease state is the metabolic syndrome” can also be set. Returning to FIG. 14, the description will be continued.

Subsequently, the output unit 102 of the medical assistance device 10 transmits the option of the behavior target determined in Step S200 to the patient terminal 20 (S201). The patient terminal 20 displays the option of the behavior target received from the medical assistance device 10 on the screen, and receives the selection of the behavior target to be handled from the patient (S202). The patient terminal 20 transmits the selected behavior target to the medical assistance device 10 (S203). The reception unit 103 of the medical assistance device 10 receives the selected behavior target, and stores the selected behavior target in the patient information 100a (S204).

Subsequently, the output unit 102 of the medical assistance device 10 refers to the questionnaire definition information 100d, and transmits the data of the questionnaire stored in the questionnaire definition information 100d to the patient terminal 20 (S205). The patient terminal 20 displays the questionnaire received from the medical assistance device 10 on the screen, and receives an input of the answer to questionnaire from the patient (S206). The patient terminal 20 transmits the answer to questionnaire received from the patient to the medical assistance device 10. In addition, the patient terminal 20 transmits the biological information acquired from the wearable device worn by the patient himself/herself (S207). The reception unit 103 of the medical assistance device 10 receives the answer to questionnaire and the biological information, and stores both of these in the patient information 100a (S208). Subsequently, the calculation unit 104 of the medical assistance device 10 calculates the score relating to the achievement degree of the behavior target from the answer to questionnaire and/or the biological information (S209).

In addition, the output unit 102 refers to the advice definition information 100g, and selects an advice to be output to the patient by comparing the answer to questionnaire, the score, and/or the biological information with an output condition (S209). The output unit 102 may be configured to select a short-term advice (for example, a daily review) by comparing the answer to questionnaire, the score, and/or the biological information of the previous day with the “output condition” of the advice (daily) definition information 100g-1. In addition, the output unit 102 may be configured to select a medium-term advice (for example, a weekly review) by comparing the answer to questionnaire, the scores, and/or the biological information of the past one week with the “output condition” of the advice (weekly summary) definition information 100g-2 for each predetermined day of the week (for example, Friday or the like). When the medium-term advice is selected, the output unit 102 may be configured to compare the transition or the average value of the scores of the past one week with the “output condition.” In addition, the output unit 102 may be configured to select a long-term advice (for example, a monthly review) by comparing the answer to questionnaire, the scores, and/or the biological information of the past one month with the “output condition” of the advice (monthly summary) definition information 100g-3 for each predetermined day (for example, the end of the month or the like) in one month. When the long-term advice is selected, the output unit 102 may be configured to compare the transition or the average value of the scores for the past one month with the “output condition.”

Subsequently, the output unit 102 transmits the calculated score and the selected advice to the patient terminal 20 (S210). The patient terminal 20 displays the score and the advice on the screen (S211). In addition, the patient terminal 20 may further display the current or future image of the appearance of the patient, the current or future image of the organ, trivia relating to the medical information, an image indicating the transition of the personal data of the patient, an image indicating the transition of the scores, various quizzes, and the like (S212). Data required for displaying these images may be transmitted from the medical assistance device 10 to the patient terminal 20 in the process procedure in Step S210.

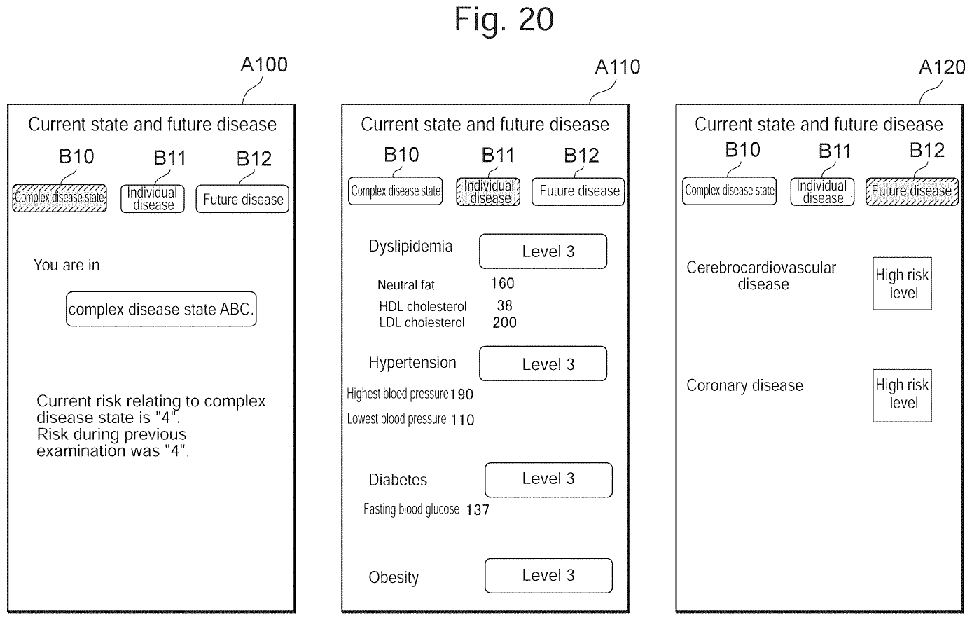

The doctor terminal 30 receives an instruction from the doctor, and requests the medical assistance device 10 to refer to the behavior target of the patient, the answer to questionnaire, the score relating to the achievement degree of the behavior target, and the advice (S220). The output unit 102 of the medical assistance device 10 transmits the behavior target selected by the patient, the answer to questionnaire, the score relating to the achievement degree of the behavior target, and the advice in the medical assistance device 10, to the doctor terminal 30 (S221). The doctor terminal 30 displays the behavior target, the answer to questionnaire, the score, and the advice which are received, on the screen (S222). In this manner, the doctor can check the daily behavior of the patient at any timing.

<Screen Display Example> (Patient Terminal) FIGS. 20 to 24 are views showing screen display examples of the patient terminal 20. Screens A100 to A120 in FIG. 20 are examples of screens for displaying current disease states of the patient himself/herself and the like. When a button B10 is pressed, the screen is transitioned to the screen A100 for displaying a disease name of the complex disease state. When the button B11 is pressed, the screen is transitioned to the screen A100 for displaying the disease name of the individual disease and the state (severity) of the individual disease. When the button B12 is pressed, the screen is transitioned to the screen A100 for displaying the disease name and the disease risk of the suffering patient. For example, the screen A100 to the screen A120 correspond to the screen displayed in the process procedure in Step S111 in FIG. 13.

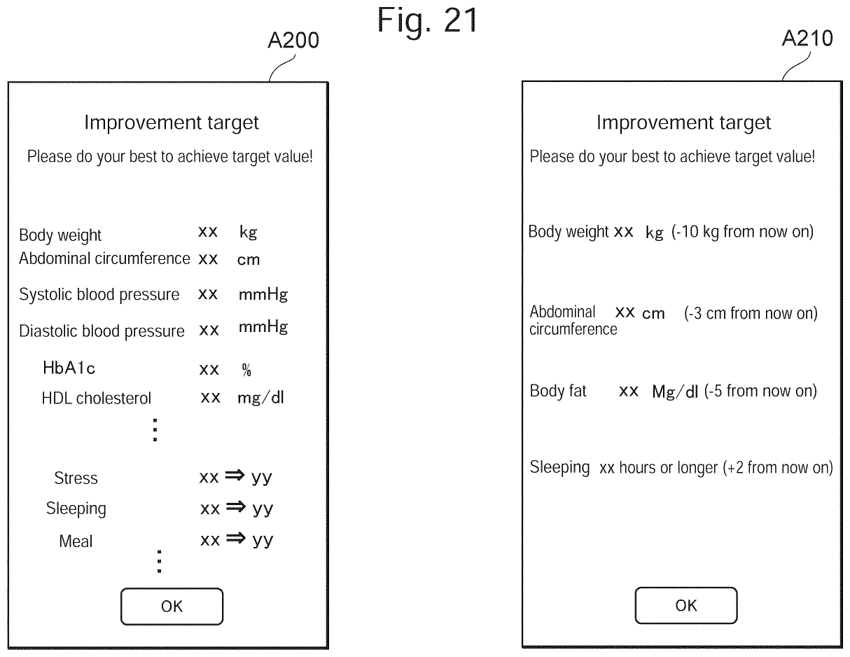

A screen A200 and a screen A210 in FIG. 21 show an example of the screen on which the improvement target is displayed. The screen A200 is a display example when all items of the improvement target are displayed, and the screen A210 is a display example when the display is limited to some items in order to facilitate understanding of the patient. For example, the screen A200 and the screen A210 correspond to the screen displayed in the process procedure in Step S118 in FIG. 13.

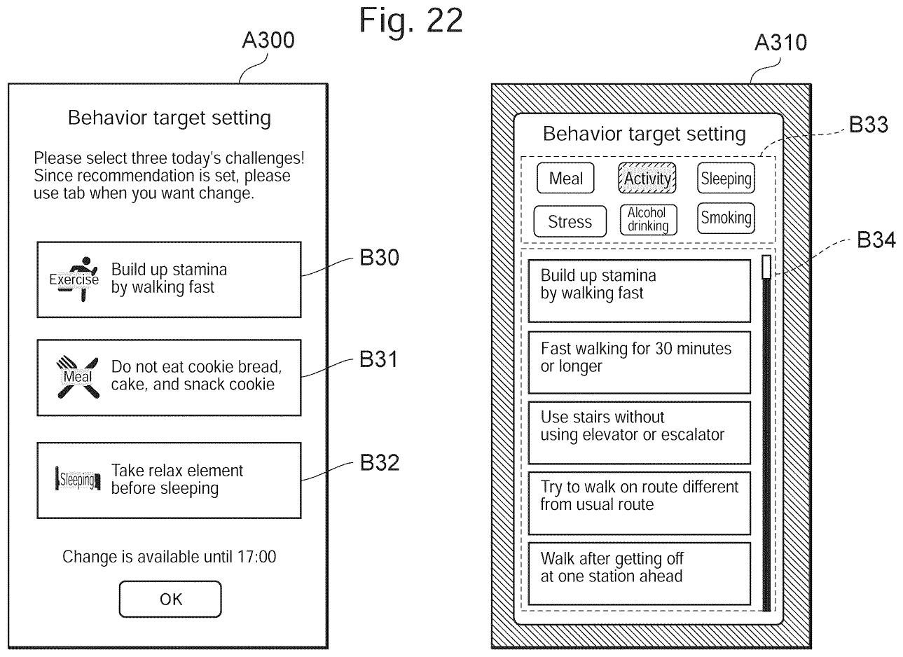

A screen A300 and a screen A310 in FIG. 22 show an example of the screen for receiving the settings of the behavior target. In the screen A300, maximum three behavior targets can be set, and when buttons B30 to B32 are pressed, the screen A310 for displaying the options of the behavior targets shows the transition. The screen A310 includes a region B33 for displaying a button for selecting the category of the behavior target, and a region B34 for displaying a list of options of the behavior target corresponding to the selected category. When the button B30 is pressed, the screen A310 shows the transition, and the behavior target selected in the region B34 on the screen A310 is reflected in the button B30 on the screen A300. Similarly, when the button B31 is pressed, the screen A310 shows the transition, and the behavior target selected in the region B34 of the screen A310 is reflected in the button B31 on the screen A300. For example, the screen A300 and the screen A310 correspond to the screen displayed in the process procedure in Step S202 in FIG. 13.

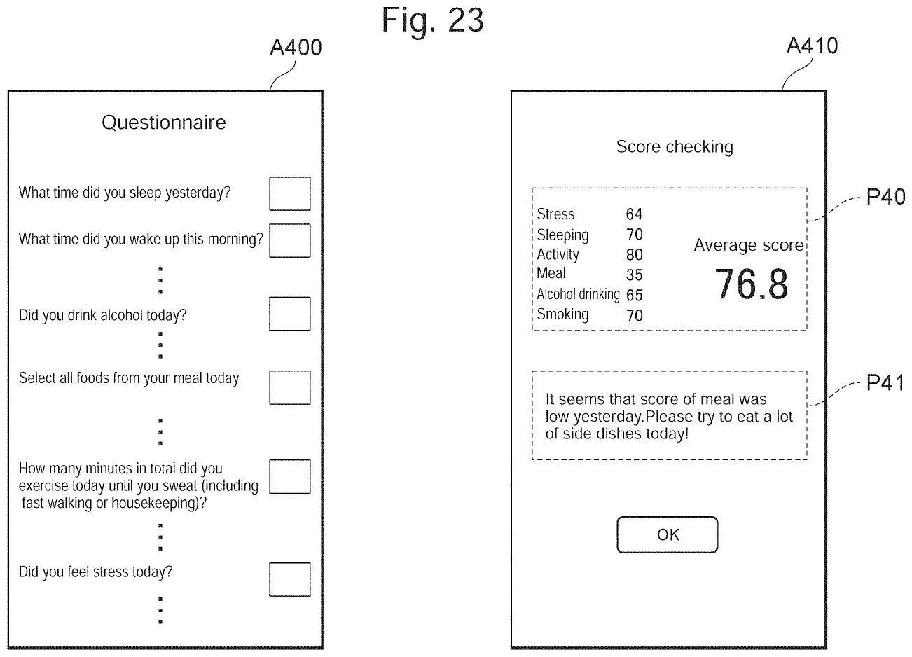

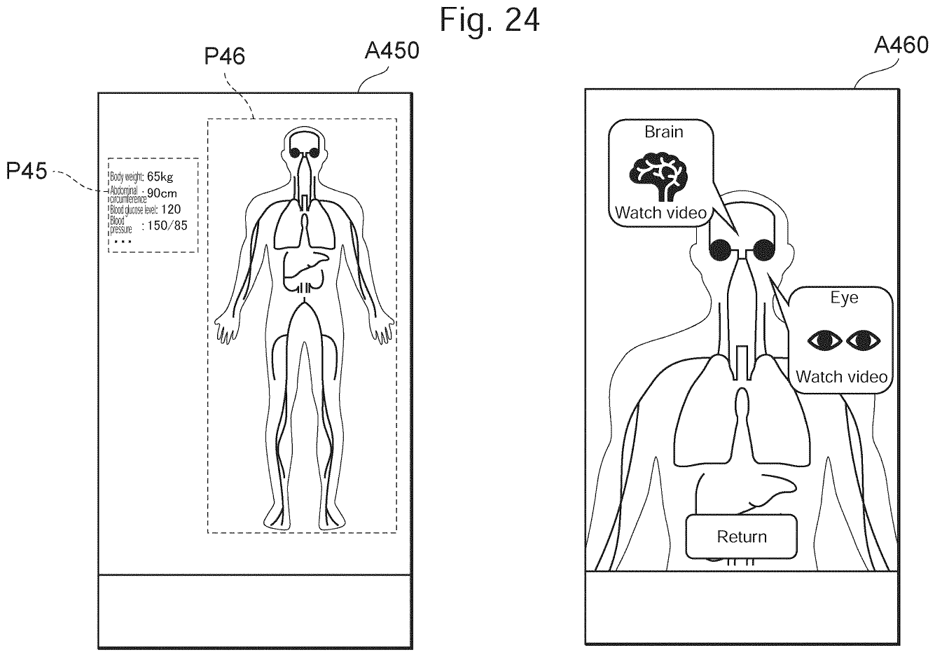

A screen A400 in FIG. 23 shows an example of the screen for inputting the answer to questionnaire. For example, the screen A400 corresponds to the screen displayed in the process procedure in Step S206 in FIG. 13. A screen A401 in FIG. 23 shows an example of the screen for displaying the score relating to the achievement degree of the behavior target. In a region P40, the score relating to the achievement degree of the behavior target is displayed for each category. As shown in a region P41 on the screen A410, the output unit 102 of the medical assistance device 10 may be configured to display the advice for the score on the screen of the patient terminal 20. The displayed advice may be an advice relating to the category having the lowest score. In addition to the daily advice, a configuration may be adopted such that the advice as a weekly summary may be displayed on a predetermined day of the week (for example, Friday or the like), and the advice as a monthly summary are be displayed on a predetermined day of the month (for example, the end of the month or the like). For example, the screen A401 corresponds to the screen displayed in the process procedure in Step S211 in FIG. 13.

FIG. 24 is an example of the screen for graphically displaying the disease risk of the patient. In the present embodiment, the medical assistance device 10 may be configured to calculate the risk of a future disease occurring in each organ of the patient, based on the personal data of the patient, and to display the calculated result in the patient terminal 20. A region P45 for displaying the examination data and an entire view P46 of the body are displayed on a screen A450. In the entire view P46 of the body, an organ that has a possibility of the future disease is emphasized and displayed. An example on the screen A 450 shows a possibility of the disease occurring in both eyes in the future. A screen A460 shows a state where a part of the entire view of the body is enlarged and displayed.

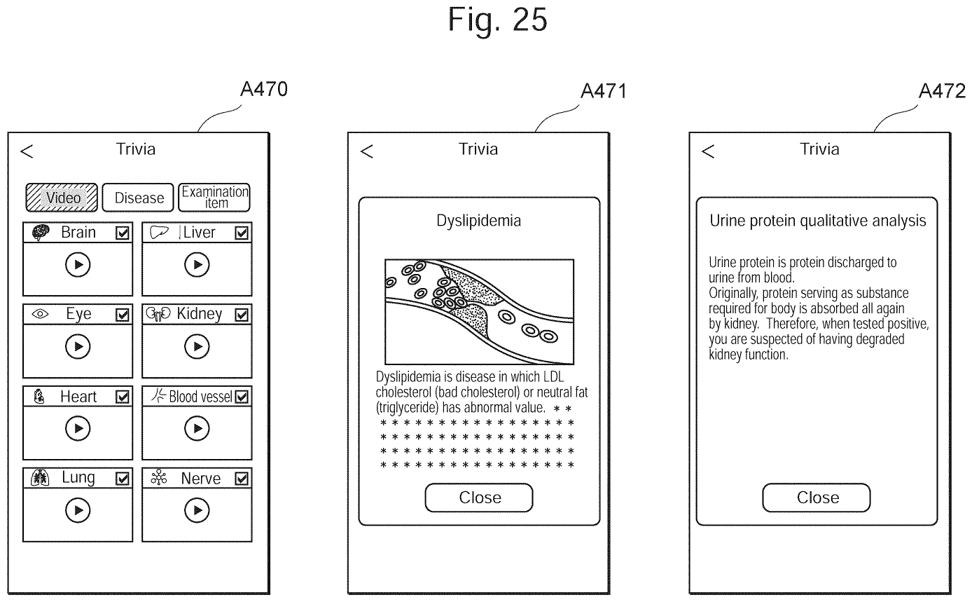

FIG. 25 shows an example of the screen for displaying “trivia” that is specific medical information on the disease or the examination item relating to each organ. The trivia relating to the organ selected on an organ list screen A470 is displayed as in trivia screens A471 and A472.

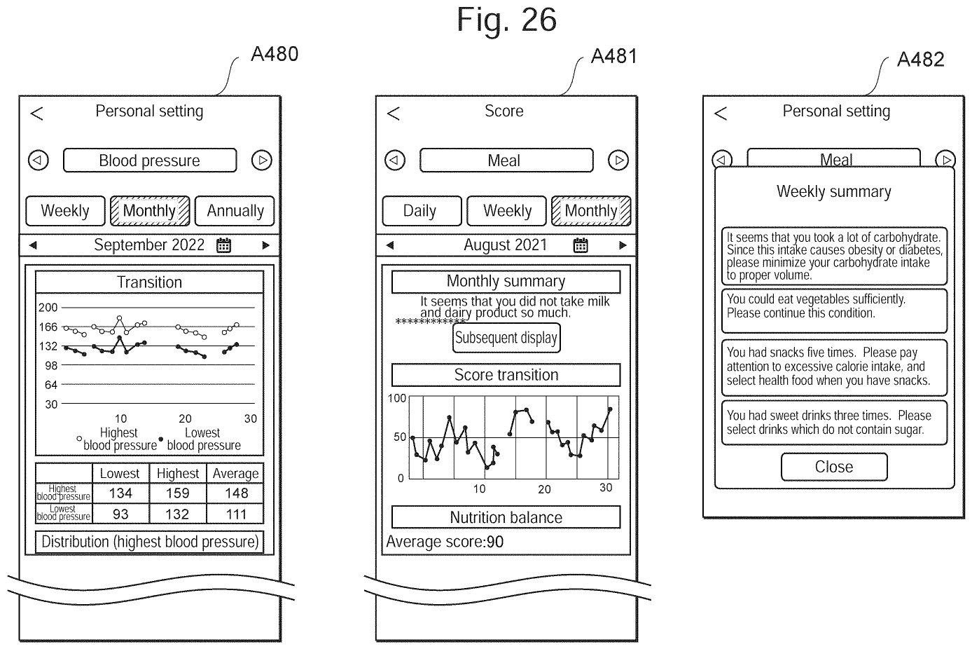

FIG. 26 shows an example of the screen for displaying a transition of the personal data of the patient. A screen A480 displays a transition of the blood pressure, and a screen A481 displays a transition of the score relating to meal. A screen A482 displays a weekly summary generated based on the content of the answer to questionnaire which is input by the patient with regard to meal.

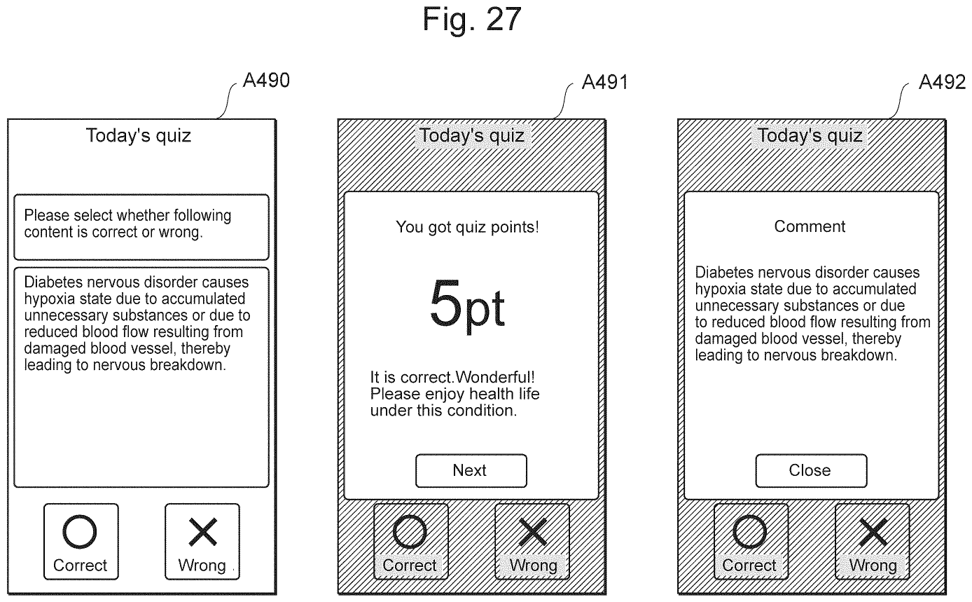

FIG. 27 shows an example of the screen relating to the quiz. A screen A490 is a screen to which the answer to quiz is input. When the patient correctly answers the quiz, points are assigned to the patient as displayed on a screen A491. The screen A492 displays a comment on the quiz.

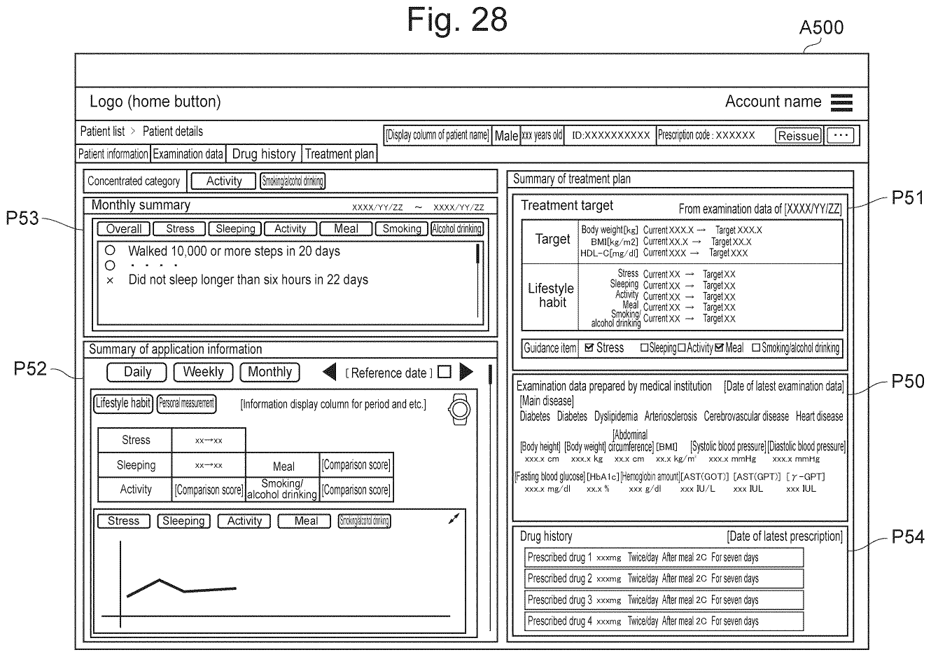

(Doctor Terminal) FIGS. 28 to 30 are views showing screen display examples of the doctor terminal 30. A screen A500 in FIG. 28 is an example of the screen for displaying a list of information relating to the patient. The examination data of the patient is displayed in a region P50. The improvement target (improvement target value for the examination value and improvement target value of the score relating to the lifestyle habit) is displayed in a region P51. A daily change in the score relating to the achievement degree of the behavior target is displayed in a region P52. In a region P53, a monthly summary of the result of the answer to questionnaire or a monthly summary of the data acquired from the wearable device of the patient is displayed. A drug history of the patient is displayed in a region P54.

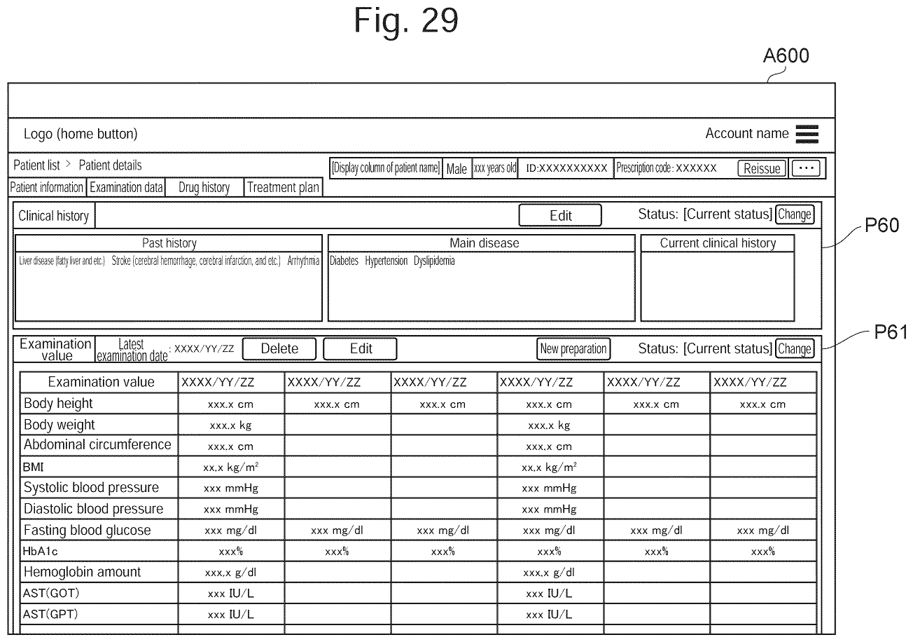

A screen A600 in FIG. 29 is an example of the screen for displaying the clinical history and the examination data in detail. The clinical history of the patient is displayed in a region P60. The examination data of the patient is displayed in a list in a region P61.

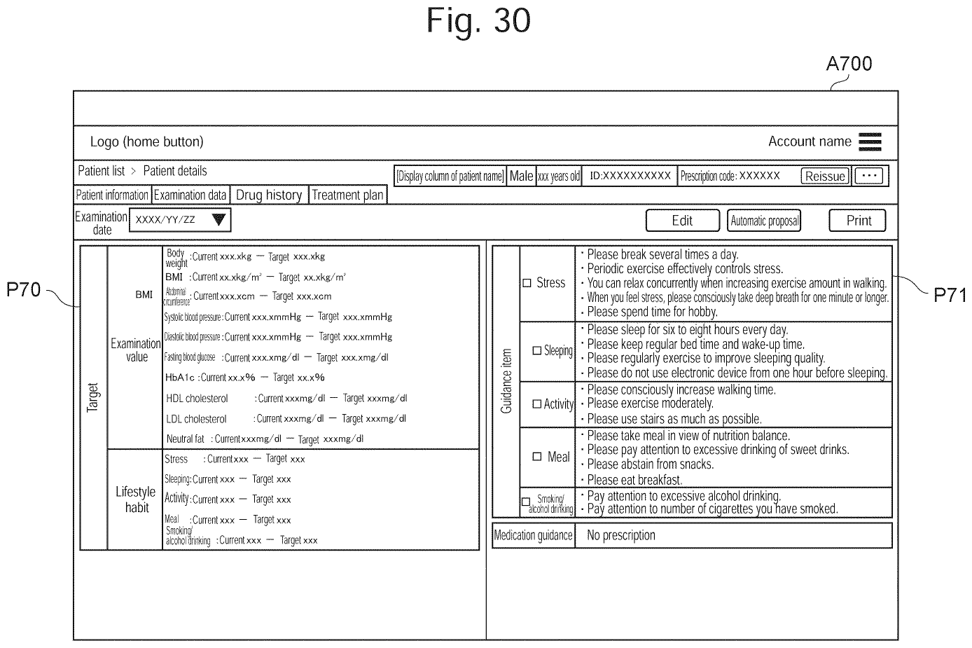

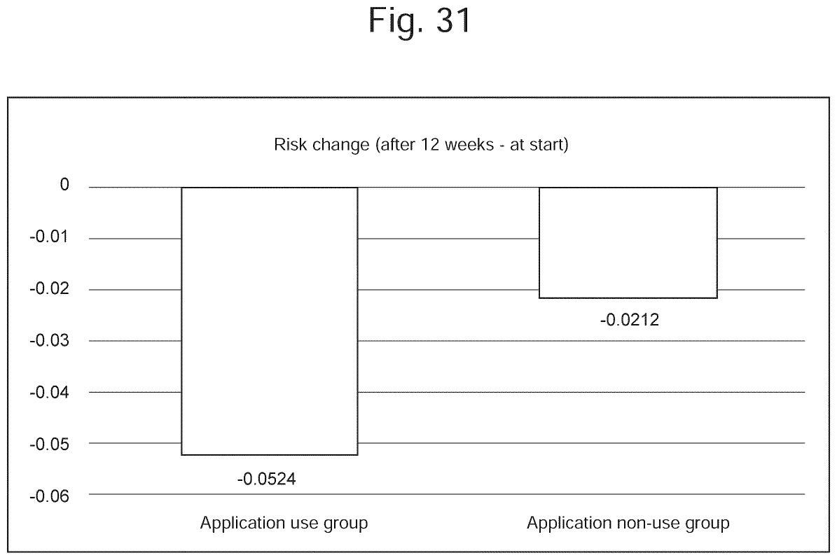

A screen A700 in FIG. 30 is an example of the screen for displaying the improvement target in detail. In a region P70, the improvement target of the patient is displayed after being divided into the improvement target value for the examination value and the improvement target value of the score relating to the lifestyle habit. The doctor can correct the improvement target displayed in the region P70. A region P71 is the screen on which the content of the guidance to the patient is recorded. When examining the patient, the doctor can record the content transmitted to the patient in the region P71.

<Experimental Results> A left graph in FIG. 31 shows a change in the risk value relating to the complex disease state calculated from the examination data of the patient in accordance with the above-described determination method 2, for the patient in the complex disease state, whose lifestyle improvement is attempted by using the medical assistance system 1 according to the present embodiment, when the lifestyle improvement starts and after 12 weeks of the lifestyle improvement. In addition, a right graph in FIG. 31 shows a change in the risk value relating to the complex disease state calculated from the examination data of the patient in accordance with the above-described determination method 2, for the patient in the complex disease state, whose the lifestyle improvement is attempted without using the medical assistance system 1 according to the present embodiment, when the lifestyle improvement starts and after 12 weeks of the lifestyle improvement. According to the experimental results, it can be understood that the patient in the complex disease state, whose lifestyle improvement is attempted by using the medical assistance system 1 according to the present embodiment, has an improved risk value.

<Summary> According to the embodiment described above, a configuration is adopted such that the disease state of the patient is determined for the individual diseases forming the complex disease, based on the personal data of the patient, the risk of the related disease is evaluated. When the combination of the disease states of the individual diseases is the complex disease state, the improvement target to be achieved by the patient to improve the complex disease state is output not only in a form of a long-term target but also in a form of a short-term target. In addition, the embodiment aims to score the daily life, based on the input questionnaire or biological information, and a configuration is adopted such that short-term (daily) evaluation and advice can be provided. In this manner, even in a state where a plurality of diseases are complexed, it is possible to propose a target value and a target setting which have to be enhanced compared to a case of a single disease. Accordingly, it is possible to provide a technology which can properly assistance treatment intervention of the patient. In addition, in the related art, it is common to perform treatment for each individual disease in spite of a state where the diseases complicatedly and concurrently occur in actual clinical practice or a state where there is a high possibility that the diseases concurrently occur soon, and complex treatment for the complex disease is not performed. However, in the present embodiment, the medical assistance device 10 presents the proper improvement target corresponding to the complex disease state, based on the personal data of the patient. Therefore, efficient treatment can be performed by obtaining continuous information on the daily life.

In addition, in the present embodiment, a configuration is adopted such that the improvement target is output on a premise that the patient is in the complex disease state. In this manner, the improvement target which can efficiently treat the complex disease state can be output in view of the weight (severity) of each individual disease forming the complex disease state, instead of a simple combination of the improvement targets for each individual disease.

In addition, the behavior target for achieving the improvement target is set, and the score corresponding to the answer to questionnaire relating to the set behavior target is displayed every day. Accordingly, the patient can review his/her own behavior everyday, and can maintain a motivation toward target achievement.

In addition, the personal data of the patient, the improvement target, the behavior target, the answer to questionnaire, and the like are also shared with the doctor who treats the patient. Therefore, the doctor can understand a handling status of the patient toward the improvement target, and can utilize the handling status for the subsequent examination.

The embodiments described above are provided for facilitating the understanding of the present invention, and are not intended to limit the interpretation of the present invention. The flowchart, the sequence, the elements provided in the embodiments, the disposition thereof, the materials, the conditions, the shapes, the sizes, and the like which are described in the embodiments can be appropriately modified without being limited to the examples. In addition, the configurations described in the different embodiments can be partially replaced or combined with each other.

In addition, in the embodiment described above, the patient terminal 20, the output unit 102 that outputs the data to the patient terminal 20, and the reception unit that receives various types of input from the patient terminal 20 may be respectively referred to as a second terminal, a second output unit, and a second reception unit. In addition, the doctor terminal 30, the output unit 102 that outputs the data to the doctor terminal 30, and the reception unit that receives various types of input from the doctor terminal 30 may be respectively referred to as a first terminal, a first output unit, and a first reception unit. In addition, in the embodiment described above, although the output unit 102 is configured to output a magnitude of the risk, the score, and the level of the severity by using numerical values, the present disclosure is not limited thereto. For example, a configuration may be adopted such that the output is performed by using a color (for example, red (high severity degree) to blue (low severity degree)) and the like or a symbol (for example, “A” (maximum score) to “Z” (minimum score) and the like) instead of changing the magnitude of the numerical value. In this case, the display control unit 202 of the patient terminal 20 may be configured to display a yellow icon on the screen A100 in FIG. 20 instead of displaying “the risk is “4”.” In addition, the display control unit 202 of the patient terminal 20 may be configured to display an alphabet on the screen A410 in FIG. 23 instead of the score.

### REFERENCE SIGNS LIST

1: medical assistance system
10: medical assistance device
11: processor
12: storage device
13: communication IF
14: input device
15: output device
20: patient terminal
30: doctor terminal
100: storage unit
100a: patient information
100b: individual disease definition information
100c: complex disease/target definition information
100d: questionnaire definition information
100e: score definition information
100f: behavior target definition information
101: acquisition unit
102: output unit
103: reception unit
104: calculation unit
200: storage unit
201: reception unit
202: display control unit
300: storage unit
301: reception unit
302: display control unit

1. A medical assistance device comprising:
an acquisition unit configured to acquire personal data including at least one of examination data and medical interview data of a user; and
an output unit configured to determine a disease state of the user for an individual disease forming a complex disease, based on the personal data, and configured to output an improvement target to be achieved by the user to improve a complex disease state, when a combination of the disease states of the individual diseases or a combination of the disease state of the individual disease and the personal data is the complex disease state.

2. The medical assistance device according to claim 1, wherein the improvement target includes an improvement target value for an examination value included in the personal data.

3. The medical assistance device according to claim 2, wherein the output unit sets an improvement target for the examination value included in the personal data, based on the combination of the disease states of the individual diseases or the combination of the disease state of the individual disease and the personal data.

4. The medical assistance device according to claim 1, wherein the personal data includes data relating to a lifestyle habit of the user, and the improvement target includes an improvement target for the lifestyle habit of the user.

5. The medical assistance device according to claim 4, wherein the lifestyle habit includes at least one of stress of the user, sleeping hours of the user, an exercise amount of the user, a dietary habit of the user, a smoking amount of the user, and alcohol drinking amount of the user.

6. The medical assistance device according to claim 1, wherein the output unit outputs the improvement target obtained by inputting the personal data to a trained model having a capability of outputting an improvement target for improving the complex disease state, as the improvement target to be achieved by the user.

7. The medical assistance device according to claim 1, further comprising:
a storage unit configured to store individual disease definition information defining a correspondence relationship between the personal data and the disease state of the individual disease, and target definition information defining an improvement target when the combination of the disease states of the individual diseases or the combination of the disease state of the individual disease and the personal data is the complex disease state,
wherein the output unit determines the disease state of the user by referring to the individual disease definition information, acquires the improvement target when the combination of the disease states of the individual diseases or the combination of the disease state of the individual disease and the personal data is the complex disease state, by referring to the target definition information, and outputs the acquired improvement target as the improvement target to be achieved by the user.

8. The medical assistance device according to claim 1, further comprising:
a first reception unit configured to receive a behavior target to be handled by the user from the user, from a plurality of options relating to a behavior target for achieving the improvement target,
wherein the output unit outputs the behavior target to be handled by the user, which is received by the first reception unit, to a terminal used by a doctor who examines the user.

9. The medical assistance device according to claim 8, wherein the first reception unit determines the plurality of options, based on the personal data, the improvement target, or the complex disease state of the user.

10. The medical assistance device according to claim 8, wherein the first reception unit receives an input of an answer to questionnaire relating to the behavior target to be handled by the user,
the medical assistance device further comprises a calculation unit configured to calculate a score relating to an achievement degree of the behavior target to be handled by the user, based on the answer to questionnaire received by the first reception unit, and
the output unit outputs the score calculated by the calculation unit.

11. The medical assistance device according to claim 8, wherein the behavior target includes at least one of a behavior target relating to stress improvement of the user, a behavior target relating to improvement in sleeping hours of the user, a behavior target relating to exercise amount improvement of the user, a behavior target relating to improvement in dietary habits of the user, a behavior target relating to smoking amount reduction of the user, and a behavior target relating to alcohol drinking amount reduction of the user.

12. The medical assistance device according to claim 1, wherein the output unit calculates a risk for each examination item for the individual disease forming the complex disease by using a reference value of one or more examination items relating to the individual disease and examination data for the examination item obtained by examining the user, determines that the user is in the complex disease state, when the calculated risk for each examination item satisfies a predetermined condition, and output the improvement target to be achieved by the user to improve the complex disease state.

13. The medical assistance device according to claim 12, wherein when the calculated risk for each examination item satisfies the predetermined condition, a value obtained by averaging the calculated risks for each examination item is equal to or greater than a predetermined threshold value, and
the output unit outputs the value obtained by averaging the calculated risks for each examination item, as a value indicating a risk relating to the complex disease state.

14. A medical assistance method executed by a medical assistance device, the method comprising:
a step of acquiring personal data including at least one of examination data and medical interview data of a user; and
a step of determining a disease state of the user for an individual disease forming a complex disease, based on the personal data, and outputting an improvement target to be achieved by the user to improve a complex disease state, when a combination of the disease states of the individual diseases or a combination of the disease state of the individual disease and the personal data is the complex disease state.

15. A computer-readable, non-transitory storage medium storing a medical assistance program causing a computer to execute a process comprising:
a step of acquiring personal data including at least one of examination data and medical interview data of a user; and
a step of determining a disease state of the user for an individual disease forming a complex disease, based on the personal data, and outputting an improvement target to be achieved by the user to improve a complex disease state, when a combination of the disease states of the individual diseases or a combination of the disease state of the individual disease and the personal data is the complex disease state.
